(19) United States
(12) Patent Application Publication
(10) Pub. No.: US 2021/0333257 A1
(43) Pub. Date: Oct. 28, 2021

(54) METHODS AND COMPOSITIONS INVOLVING FLAVONOLS

(71) Applicant: The Regents of the University of California, Oakland, CA (US)

(72) Inventors: Johann D. MARTINEZ, Davis, CA (US); Luca BRILLANTE, Davis, CA (US); Kaan KURTURAL, Davis, CA (US)

(21) Appl. No.: 17/269,494

(22) PCT Filed: Aug. 20, 2019
(86) PCT No.: PCT/US19/47261

§ 371 Date: Feb. 18, 2021

Related U.S. Application Data
(60) Provisional application No. 62/720,000, filed on Aug. 20, 2018.

(51) Int. Cl. G01N 33/02; A01G 17/00; G01N 21/25; G01N 1/34

(52) U.S. Cl. CPC G01N 33/025; G01N 1/34; G01N 21/251; A01G 17/005

(57) ABSTRACT

The disclosure provides methods for assessing the exposure of red grapes by measuring the amounts of 4′-OH flavonol, 3′4′-OH flavonol, and 3′4′5′-OH flavonol extracted from red grape skins. The disclosure also describes use of Partial Least Squares (PLS) regression for determining the relative percentages of 4′-OH flavonol, 3′4′-OH flavonol, and 3′4′5′-OH flavonol.

### CROSS-REFERENCE TO RELATED PATENT APPLICATIONS

The present application claims benefit of priority to U.S. Provisional Patent Application No. 62/720,000, filed Aug. 20, 2018, which is incorporated by reference for all purposes.

### BACKGROUND

In wine grape growing, canopy density is controlled either during the dormant period through pruning, or during the growing season through fruit-zone leaf removal and shoot thinning. These techniques aim mainly to control fruit load, but also to expose grapes to a higher air circulation and solar radiation. This has been associated to a reduction in fungal diseases (Chellemi and Marois, 1992), but also some desirable effects for specific winemaking targets, such as green aroma removal in Cabernet Sauvignon (Koch et al., 2012) or higher content in phenolic compounds in full bodied red wine grapes (Matus et al., 2009). However, this is not a general rule and some winemaking frameworks, such as sparkling rose, do not pursue a great phenolic content but a higher acidity and acidic fruit aromas, which may be affected by exposure due to increasing temperature (Sweetman et al., 2014). Even when moderate exposure is desired, the impact of the excess of solar radiation can be negative, resulting in flavonoid and organic acid degradation (Martínez-Lüscher et al. 2017).

Canopy size is also the focus of vineyard management due to the need of balance with fruit load. The spatial assessment of vine vigour (i.e., vegetative growth) is a widely used tool in the industry and research, especially for precision agriculture due to its relation to grape composition (Baluja et al., 2012; Cortell et al., 2007). Uneven vine vigour can be an indication of nutrient deficiency, different soil profiles, source/sink balances, different mesoclimates, or pathogens. These factors may lead to a suboptimal use of a plot (Steyn et al., 2016). Therefore, there is a strong need for tools that are able to quantify and optimize the exposure of red grapes and the assessment of canopy size.

### SUMMARY

In one aspect, the disclosure features a method for determining whether a grape is under exposed or overexposed to solar radiation. In some embodiments, the method comprises:

(a) extracting and purifying flavonols comprising 4′-OH flavonol, 3′4′-OH flavonol, and 3′4′5′-OH flavonol from grape skins;

(b) determining percentages of 4′-OH flavonol, 3′4′-OH flavonol, and 3′4′5′-OH in the extracted flavonols using a spectrophotometer and a Partial Least Squares (PLS) regression model, wherein:

if the percentage of 4′-OH flavonol content is below 4%, then the grapes are underexposed and canopy will need pruning; or

if the percentage of 4′-OH flavonol is above 10.5%, then the grapes are overexposed and need cover; or

if 3′4′5′-OH flavonol content is above 45%, then the grapes are underexposed and canopy will need pruning; or

if the percentage of 3′4′5′-OH flavonol is below 30%, then the grapes are overexposed and need cover. In some embodiments, in view of the method, grapes are covered or the canopy is pruned.

4′-OH flavonol is also referred to as kaempferol glycosides herein. 3′4′-OH flavonol is also referred to as quercetin and isorhamnetin glycosides herein. 3′4′5′-OH flavonol is also referred to as myricetin, laricitin and syringetin glycosides herein.

In some embodiments, the PLS regression model comprises an equation using the absorbance of a purified flavonol mixture at multiple wavelengths between 220 and 800 nm. In some embodiments, the PLS model calculates the concentration of each of 4′-OH flavonol, 3′4′-OH flavonol, and 3′4′5′-OH flavonol, calculates their percentage of total flavonol and plots the percentages in a ternary plot for the comparison to grapes with different exposures. In some embodiments, pruning the grape canopy comprises shoot thinning and/or leaf removal to increase exposure.

Also provided is a computer program product comprising a computer-readable storage medium containing computer program code for:

receiving a data set representing the absorbance of a mixture of flavonols measured at all wavelengths between 220 and 800 nm,

applying a Partial Least Squares (PLS) regression precalibrated model to generate percentage of the one or more flavonols selected from the group consisting of 4′-OH flavonol, 3′4′-OH flavonol, and 3′4′5′-OH, and comparing the percentage of the flavonol to a cut-off percentage.

In some embodiments, the flavonol is 4′-OH flavonol and the cut-off percentage is 4% out of total flavonol content. In some embodiments, the flavonol is 4′-OH flavonol and the cut-off percentage is 10.5% out of total flavonol content. In some embodiments, the flavonol is 3′4′5′-OH flavonol and the cut-off percentage is 30% out of total flavonol content In some embodiments, the flavonol is 3′4′5′-OH flavonol and the cut-off percentage is 45% out of total flavonol content.

In some embodiments, the computer program product further comprises code for indicating whether pruning is suggested or not based on the comparing.

### BRIEF DESCRIPTION OF THE DRAWINGS

FIG. 1 shows Changes in flavonol synthesis observed by Martínez-Lüscher et al. (2014a) in response to supplemental monochromatic UV-B radiation and to water deficit under controlled conditions (Right). *Constitutive synthesis is deduced from the flavonol content of V. vinifera cv. Tempranillo grapes under UV-B filtered solar radiation, 24/14° C. cycle (day/night) and pots watered to field capacity. Flavonol synthase (FLS) is strongly upregulated by UV-B altering the balance with the levels of flavonoid hydroxylases (F3′H and F3′5′H), leading to a higher relative abundance of the less hydroxylated forms (4′-OH and 3′4′-OH flavonols).

FIG. 2 shows the effect of formic acid concentration during the optimization of HPLC method on the quantification of flavonols (green) in Cabernet sauvignon grape skins. A concentration of 5% of formic acid in the mobile phases allows the separation of different flavonols while avoiding the co-elution with anthocyanins (pink) in the apparatus and column used in this study.

FIGS. 3A and 3B show a fish eye lens image of the canopy from the cluster perspective (A) and blue channel of the same image used in image processing to calculate the proportion of the image where the sky is visible (canopy porosity; B).

FIGS. 4A and 4B show bar graphs demonstrating the Partial Least Squares Regression (PLS) of canopy porosity from the perspective of individual clusters of Cabernet Sauvignon using the cluster position, trellis, berry characteristics, and composition of the 5 berries on the top of each cluster as predictor variables. Regression coefficient (FIG. 4A) and Variable importance in the projection (VIP; FIG. 4B) of the predictor variables are the output of a PLS regression model performed with these variables showing their capacity to predict grape exposure. A, Anthocyanins; F, Flavonols; SDM, skin dry mass; BFM, berry fresh mass.

FIGS. 5A-5D show scatterplots demonstrating the relationship between canopy porosity and flavonol content per berry and molar relative abundance of 4′-OH, 3′4′-OH and 3′4′5′-OH flavonols in 5-berry samples collected from the top of individual clusters.

FIGS. 6A-6D show scatterplots demonstrating the evolution of flavonol content per berry and the proportion of 4′-OH, 3′4′-OH and 3′4′5′-OH flavonols under ambient (0% shading factor) and under two shade nets (20% and 40% shading factor).

FIGS. 7A and 7B show scatterplots demonstrating the extinction of light by increasing canopy density (pruning weight; FIG. 7A) and linear relationship between light transmission and the inverse of pruning weights per meter of row (FIG. 7B).

FIGS. 8A-8L show scatterplots demonstrating the relationship between flavonol content per berry (FIGS. 8A, 8E, and 8I), molar relative abundance of 4′-OH (FIGS. 8B, 8F, and 8J), 3′4′-OH (FIGS. 8C, 8G, and 8K), and 3′4′5′-OH (FIGS. 8D, 8H, and 8L) flavonols and the inverse of canopy density (FIGS. 8A-8D), stem water potential (FIGS. 8E-8H), and total soluble solids (FIGS. 8I-8L) of cv. Cabernet Sauvignon (Cab) trained as high quadrilateral cordons and sprawling canopy (Quad Sprawl; Blue), and cv. Merlot (Mer) trained as bilateral cordons and vertical shoots positioning (VSP; Red).

FIG. 9 shows steps in the spectrophotometric procedure for the determination of flavonol profile including: 1, skin the grapes; 2, place skins in extracting solution; 3, let extract for at least 1 hour; 4, remove the skins by centrifuging of filtering; 5, place 1 mL of extract onto the column; 6, let the sample be absorbed; 7, pore 7 mL of water in the column (discard); 8, use the plunger to make the water pass through; 9, pore 5 mL of methanol onto the column; 10, use the plunger to make the methanol to elute flavonols from the column; 11, collect the methanol extract into a sample tube; 12, dilute the extract into a cuvette to the right concentration; and 13, scan the cuvette for 220 to 800 nm and process the data according to the PLS regression algorithm.

FIG. 10 shows a block diagram of an example computer system 100 usable with system and methods according to embodiments of the present disclosure.

FIG. 11 shows the empirical relationship between flavonol profiles determined by HPLC-DAD of single berries (gray squares; n=215). Lines marked with B delimit the threshold beyond which 95% of the underexposed (graded through visual assessment) grapes are placed for %4′-OH flavonols (<4%) and %3′4′5′-OH flavonols (>45%). Lines marked with B delimit the threshold beyond which 95% of the underexposed (graded through visual assessment) grapes are placed for %4′-OH flavonols (>10.5%) and %3′4′5′-OH flavonols (<30%).

FIG. 12 shows that the three quercetin glycosides most frequently found in V. vinifera grapes and present in the standard used were not separated under all conditions.

FIGS. 13A-13D show that the flavonol profile is intimately related to the accumulated global radiation received by grapes and this response may be exacerbated when degradation takes place. Correlation between accumulated global radiation from beginning of ripening to maturity and the percent of kaempferol (A) or total flavonols per berry (B). Correlation between the percent of kaempferol and total flavonols per berry (C). Berry flavonol content (bars) and profile (pies) of grapes from interior, exposed, moderately, and severely overexposed clusters (D). Circles for sprawling canopy and triangles for vertical-shoot-positioned trellis. Dashed lines are breaking points determined through segmented regression.

FIG. 14 shows the cluster temperatures measured with an infrared thermometer on fully exposed clusters in a Cabernet sauvignon vineyard with vertical-shoot-position trellis with NE to SW row orientation in Oakville, Calif. on 11 Sep. 2017.

FIGS. 15A-15N show the relationship between estimated canopy porosity and leaf area index (LAI) and flavonol content per berry (A,B), the percent of kaempferol (C,D), the percent of quercetin (E,F), the percent of myricetin (G,H), the percent of isorhamnetin (I,J), the percent of laricitin (K,L), and the percent of syringetin (M,N) in 5-berry samples collected from the top of individual clusters. Circles for sprawling canopy and triangles for vertical-shoot-positioned trellis.

FIG. 16 shows the minima, average, and maxima of yield components, water status, total soluble solids, anthocyanin content, flavonol content and profile, and climatic conditions (from 15th May to 15th October) of two experimental sites used to study variations of vigor, water status and developmental stage on flavonol content and profile (FIGS. 17A-17L).

FIGS. 17A-17D show the Kriged maps of the percent of kaempferol in 2D (A) and 3D (B), maps of satellite-sensed NDVI (C), and relationship between % kaempferol and NDVI (D) in a cv. Merlot vineyard trained as bilateral cordons and vertically shoot positioned. (A) Root mean squared error associated with the kriging procedure is 0.8% kaempferol estimated with leave-one out cross-validation. Elevation is exaggerated twice in 3D map (B) to enhance topographic variation within the vineyard. Coordinates are EPSG:32610. Black dots in the map are the centroid of the 5-vine experimental units. (B) Overlap of map in (A) on a Google Earth background, view from the South-East (upper) and South-West corner (lower). (C) Three meters resolution, satellite-sensed NDVI map dated 2016 Jul. 14. Black dots in map C are the centroid of the 5-vine experimental units. Coordinates in (A,C) are EPSG:32610, consequently units are in meters. Background map in (A,C) is an USGS high resolution aerial image dated 2014 Aug. 11.

FIG. 18 shows the dormant pruning weight and flavonol composition at harvest in a cv. Merlot, Paso Robles, Calif., United States, vineyard from vines homogeneously distributed and grouped by their pruning weights and canopy management treatments.

### DETAILED DESCRIPTION OF THE EMBODIMENTS

### I. Introduction

Exposure to solar radiation is a big driver for grape composition. Flavonols are an important part of acclimation to high solar radiation in plant tissues. Their synthesis is unregulated by UV-B radiation, leaving an unmistakable fingerprint on their diversification according to their substituents in the B-ring of the flavonoid skeleton.

The disclosure is directed to using the flavonol profile of a grape (e.g., red grape plant) as a biomarker (i.e. indicator) to assess the overall exposure of red wine grapes to solar radiation. The percentage of each flavonol in the flavonol profile can be used as an indication of whether the red grape canopy needed to be pruned (leaf removal or shoot thinning) in order to optimize grape composition, or alternatively, whether the grape canopy did not protect grapes and corrective action needs to be taken next season. Changing trellis or row direction or use shade nets are options to reduce the exposure of grapes each with different costs and implications. As discussed herein, experiments were conducted to relate canopy porosity with flavonol profile, to follow flavonol profile development under solar radiation exclusion treatments, and to relate flavonol profile to natural and induced variability in commercial vineyards. Results show a strong relationship between exposure and the three components of flavonol profile, increasing the proportion of 4′, 3′4′ in detriment and 3′4′5′ hydroxylated (—OH) flavonols. However, whereas the proportion of 3′4′ and 3′4′5′-OH flavonols were sensitive to the progress of ripening, the proportion of 4′-OH flavonols remained stable for a wide period and range of total soluble solids. The proportion of 4′-OH flavonols was inversely related to canopy size (i.e., pruning weight) and was responsive to cultural practices such as shoot thinning and leaf removal. In addition, this biomarker was not sensitive to water status or different soluble solids levels, which typically have a strong effect on anthocyanin profile. Together, these results demonstrate the reliability and the usefulness of the proportion of 4′-OH flavonols as a biomarker to assess the effectivity of vineyard practices or spatial variability in relation to canopy size or grape exposure to solar radiation. The proportion of 3′4′5′-OH flavonols can be useful also due to their negative correlation to exposure. Even though 3′4′5′-OH flavonols are not as reliable as 4′-OH flavonols, 3′4′5′-OH flavonols are present in higher proportions and are easier to determine by either HPLC or spectrometry. The possibility of assessing grape solar radiation interception of pools or single grape berries opens the possibility of establishing the relationship between solar radiation and grape quality traits by future studies reducing the need of taking as many light measurements or canopy porosity.

This disclosure makes more accessible the determination of flavonols profiles (proportion of each kind of flavonols) in anthocyanin rich tissues either through HPLC-DAD method fine-tuning, or through UV-VIS multi=−wavelength spectrometry and a PLS regression model. We also aimed to propose the proportion of 4′-OH flavonols as a biomarker of the integrated solar radiation received by grapes and test its reliability. In the results, proportion of 4′-OH flavonols was more reliable predictor of canopy porosity than any other predictor variable, including visual assessments of sun damage. This reliability was based on its stability through hang time, when overall flavonols content was decreasing, and the lack of significant effect of water status.

The use of the proportion of 4′-OH flavonols was exemplified by the estimation of grape exposure in management zones resulting from proximal sensing and cultural practices aiming to increase the exposure of the grapes or balancing vine vigor. Given the widely extended use of HPLC-DAD methods to determine anthocyanin profile in research and the use of spectrometry in grape growing industry, we provided the principles to understand and methods to determine flavonol profiles and use them as biomarker of solar radiation interception by grapes.

As described in detail further herein, the disclosure includes methods of determining whether to prune a red grape canopy by determining the percentages of various flavonols (e.g., 4′-OH flavonol, 3′4′-OH flavonol, and 3′4′5′-OH flavonol) extracted from red grape skins and comparing the determined percentages to threshold values to assist in determination of solar radiation status of grape plants for grape harvest management.

### II. Flavonoids

Flavonoids are one of the most versatile metabolites in plants. For instance, they play a key role in promoting sexual reproduction of plants, enabling a normal development of pollen, and making flowers noticeable to pollinators or fruits to animals that contribute to seed dispersal (Koes et al., 1994). They play also an important role in cross-talk between plants and microorganisms, as they are exuded into the rhizosphere (Cooper, 2004; Lagrange et al., 2001). Flavonoids are remarkable for their inhibitory activity on inter-species seed germination and fungal pathogens (Kong et al., 2004). Due to their strong radical scavenging activity, flavonoids are key in the protection of the plant biochemical machinery from oxidative stress. For instance, flavonols and anthocyanins are involved in the mitigation of water stress (Nakabayashi et al., 2014). In the case of flavonols, they are accumulated in epidermal cells of plant tissues in response to light, especially UV, filtering the most novice part of the solar spectrum (Kolb et al., 2001; Martínez-Lüscher et al., 2013b).

Within each class of flavonoids, certain structural variation exist in their basic 15-carbon skeleton, leading to different physicochemical properties. In the case of flavonols and anthocyanins of wine grape (Vitis vinifera), they often present different substituents in their 3 position of the C-ring and the 3′ and 5′ positions of the B-ring. The most frequent substituent in 3 position of C-ring is glucose and in the case of anthocyanins, this substituent is crucial for its stability, color, and increase their hydrophilic characteristic (Vogt and Jones, 2000). In the case of flavonols, aglycones are reported in some cases in grape skin extracts, but often are reported in wines, suggesting that they remain stable after hydrolysis (Castillo-Munoz et al., 2007). In other fruit species, less common sugars and di-glucosides are more frequently found as substituents of in 3 position of the C-ring (Acevedo de la Cruz et al., 2012). In turn, sugar substituents can be acylated, mainly with acetyl or a phenolic hydroxyl groups. This is performed enzymatically and increases the solubility of flavonoids in various media, as well as their stability and their antioxidant capacity (Chebil et al., 2006; Zhao et al., 2017).

Anthocyanins are colorful compounds and hydroxyl and methyl group substituents in 3′ and 5′ positions of the B-ring give place to five kinds of compounds (pelargonidins, cyaniduns, peonidins, delphinidins, petunidins, and malvidins), which by themselves can give place to a range of colors from orange to purple in plant tissues (Dixon et al., 2013). Although flavonol are colorless, their substituents in the B-ring may also lead to different optical properties appreciable in the absorbance in the UV range. For both anthocyanins and flavonols, there is a relationship between the substituents in these positions and the chemical behavior of anthocyanins and flavonols. The addition of hydroxyl groups in 3′ and 5′ positions of the B-ring result in a great increase in oxidation potential as determined by cyclical voltammetry (Arroyo-Currás et al., 2016), and in vitro antioxidant capacity of flavonols, anthocyanins, and flavan-3ols (Tabart et al., 2009).

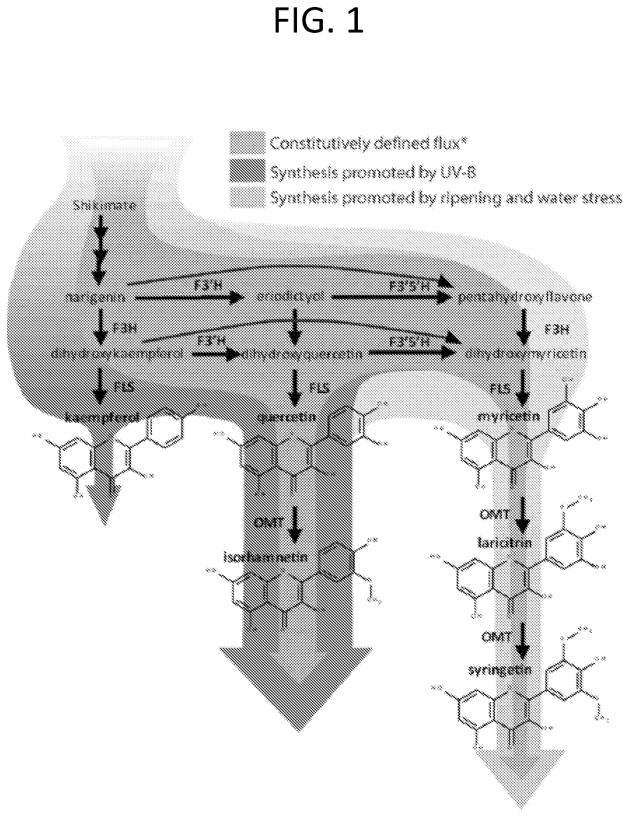

Grapes varieties constitutively present a wide range of flavonoids profiles depending their substituents in 3′ and 5′ position (Mattivi et al., 2006). However, most full-colored red wine grapes have a highly 3′ and 5′ substituted profile (rich in anthocyanins and flavonols with hydroxyl or methyl groups in 3′ and 5′ position of the B-ring). For instance, Cabernet Sauvignon, Shiraz, or Tempranillo are examples of highly 3′ 5′ substituted profiles and Sangiovese and Pinot noir are examples of poorly 3′ 5′ substituted profiles, similar to table grapes (Mattivi et al., 2006). Hereby, anthocyanin and flavonol profiles are highly determined genetically and are controlled and inherited as a quantitative loci trait (Fournier-Level et al., 2011; Malacarne et al., 2015). In fact, these profiles have been proposed as an authentication tool for wines (Hermosín-Gutiérrez et al., 2011). Ultimately, profiles are determined by the proportion in the levels of expression of the gene(s) encoding flavonoid 3′ 5′ hydroxylase (F3′5′H) and the synthetic enzyme(s) controlling the synthesis of each flavonoid (i.e., UFGT for anthocyanins and FLS for flavonols) (Castellarin and Di Gaspero, 2007; Martínez-Lüscher et al., 2014a). However, there also a strong environmental regulation of this trait. Water deficit appears to be involved in the regulation of flavonoid 3′ 5′ hydroxylase (F3′5′H), an enzyme responsible for the hydroxylation of dihydrokaempferol and dihydroquercetin into dihydromyricitin (Kaltenbach, 1999; FIG. 1), leading to higher degree of hydroxylation of anthocyanins and flavonols under water deficit (Castellarin et al., 2007; Martínez-Lüscher et al., 2014a). Exposure to solar radiation, especially UV-B radiation, upregulates the expression of flavonoid 3′ hydroxylase (F3′H), the enzyme responsible for the hydroxylation of dihydrokaempferol into dihydroquercetin, which may shift anthocyanins and flavonols to some extent towards a profile richer in compounds using dihydroquercetin as precursors.

In previous research, Martínez-Lüscher et al. (2014b) and Martínez-Lüscher et al. (2014a) proposed a mechanistic hypothesis for the constitution of anthocyanin and flavonol profile under UV-B radiation and water deficit. Hereby, grapes from fruit-bearing cuttings grown under natural light filtered through a glasshouse (99.9% and 15% of UV-B and UV-A removal, respectively) responded linearly in exposure time to an artificial source of UV-B. This response corresponded to an increase in flavonol concentration (R²=0.43), decrease in the proportion of 3′4′5′-OH (R²=0.70; negative correlation), and increase in the proportion of 3′4′-OH (R²=0.67) and 4′-OH (R²=0.82) flavonols (Martínez-Lüscher et al., 2014b). The coefficient of determination of 4′-OH flavonols was further improved (R²=0.95) by accounting radiation before and after veraison separately, and post veraison radiation appeared 4.17 times more effective than pre-veraison dose. The study of the interactive effects of UV-B with water deficit revealed that UV-B did not upregulated all genes involved in flavonoid biosynthesis in the same way. Whereas genes encoding for enzymes upstream in the pathway may present a nearly 2-fold induction (e.g., CHS and F3′H). The induction of flavonol synthase (FLS) was 10-fold (Martínez-Lüscher et al., 2014a). This disproportion allegedly resulted in stronger competition of FLS with flavonoid hydroxylases (F3′H and F3′5′H) for flavonol substrates, which reduced their chances of being hydroxylated before being transformed into flavonols (FIG. 1), leading to a lower hydroxylation profile in flavonols but not anthocyanins. This phenomenon could be exploited as a biomarker to assess grape exposure and vine vigor, to study the effects of solar radiation, and to determine light transmission trough canopies.

The disclosure is directed to using the percentages of three flavonols (e.g., 4′-OH flavonol, 3′4′-OH flavonol, and 3′4′5′-OH flavonol) extracted from red grape skins to determine whether a red grape canopy needs to be pruned, i.e., by shoot removal and/or leaf removal. The inventors have found through experimentation that the proportion of 3′4′ and 3′4′5′-OH flavonols were sensitive to the progress of grape ripening, while the proportion of 4′-OH flavonols remained stable. The three flavonols, 4′-OH flavonol, 3′4′-OH flavonol, and 3′4′5′-OH flavonols, may be used as markers that indicate, i.e., the progress of red grape ripening, the amount of solar exposure, and the ideal red grape canopy porosity. Once determined and compared to a threshold value, the determined amount of various flavonols can be used to determine whether the grape canopy is pruned, or optionally shaded to sow ripening, or harvested to avoid over ripening.

### III. Flavonol Extraction and Purification

Flavonols can be extracted from grapes (e.g., grape skins) by either liquid extraction or solid phase extraction. In liquid extraction, a suitable organic solvent (e.g., methanol, ethanol, or ethyl acetate) can be used together with water and a trace amount of acid (e.g., hydrochloric acid). As described in Example 5, a mixture of methanol, water, and hydrochloric acid was used to extract flavonols from grape skins. Liquid extraction can be performed one or more times while adjusting the amounts of organic solvent and acid. A detection method such as mass spectrometry or spectrophotometry can be used during the extraction process to ensure that most of the desired flavonols are in the aqueous phase (e.g., water) and not in the organic phase (e.g., methanol, ethanol, or ethyl acetate). The detection method can also help in adjusting the relative amounts of organic solvent and acid. Following liquid extraction, the extract can be filtered or centrifuged to remove undesired products and further purified using solid extraction, e.g., high-performance liquid chromatography (HPLC). The extract can also be evaporated to obtain a substantially dry, solid composition comprising flavonols. As used herein, “extraction” can include a step of further purification of one or more flavonol.

Solid extraction generally involves the use of a chromatographic resin column, with separation and elution of the differentially enriched fractions from the column. Depending on the technical needs, the particular length and/or particle size of the resin column can be chosen to achieve the ideal separation. Similar to liquid extraction, a mixture of organic solvent, water, and acid can be used to elute and separate the components in the extract from the resin column. Examples of organic solvents that can be used include, but are not limited to, methanol, ethanol, ethyl acetate, chloroform, and dimethyl chloride. Those skilled in the art are familiar with the various parameters that can be adjusted during a chromatographic resin column extraction and purification in order to obtain flavonols and avoid co-elution of flavonols with other components in the extract. For example, as demonstrated in Example 6, the percentage of formic acid, as well as the elution rate, can be adjusted in order to achieve the best separation between flavonols and other phenolic compounds (e.g., anthocyanins). In some embodiments, the percentage of formic acid used to extract the flavonols is between 4-8% (e.g., 5-7%, e.g. about 6%) to achieve separation of the different target flavonols.

### IV. Quantification of Flavonol Content

Following extraction, one, two, or three flavonols selected from 4′-OH flavonol, 3′4′-OH flavonol, and 3′4′5′-OH flavonols can detected and quantified from grape skins of a grape plant. 4′-OH flavonol, 3′4′-OH flavonol, and 3′4′5′-OH flavonol can be detected as desired. In some embodiments, the quantity of flavonols is determined using a spectrophotomer to detect one or more wavelengths from a plant sample (e.g., grape skin). Methods of detecting flavonols can include methods involving spectrophotometers are described in, e.g., Lorrain, et al., Molecules 2013, 18, 1076-1100 reviewing some flavonol detection methods.

In some embodiments, the individual fractions within the flavonols group of chemical compounds (i.e. 4′-OH, 3′4′-OH, and 3′4′5′-OH flavonols) are quantified based on a spectra of wavelengths using PLS regression. Examples of quantification based on one wavelength in other contexts can be found in Bouhsain et al., 1997; Richardson et al., 2004. However, in these methods single (or few) wavelength(s) and linear regression are used to quantify the target analytes as a group (i.e. total flavonols), which according to our findings does not correlate well to solar radiation due to degradation. This is a relatively new procedure resulting from the emergence of statistical analyses related to partial least squares (PLS) regression that allows to determine not only total flavonols, but the content of each flavonol group in a mixture. Single (or few) wavelength methods use linear regression and have poor performance with autocorrelared variables such as the overlapping wavelengths of the different flavonols. On the other hand, methods using wide spectra and PLS regression can be calibrated to perform well for auto correlated variables or noisy readings or contaminated samples.

Exemplary wavelengths for generating the spectra include those wavelengths between 220 and 800 nm. In some embodiments, partial least squares (PLS) regression can be applied to spectrophotometric data from the grapes to predict percentage of 4′-OH flavonol, 3′4′-OH flavonol, and 3′4′5′-OH flavonols. In some embodiments, the PLS regression model is similar to a multivariate regression model with same number of coefficients as predictor variables, for example in the form of y=coef1*x1+coef2*x2+c for 2 variables whereas the PLS regression model for the calculation of flavonol profiles would have 550 predictor variables and 550 coefficients obtained from few dozens of independent observations. The concentration of each flavonol can be generated to calculate for each flavonol group the percentage of total flavonol content of the sample. In some embodiments, the PLS regression model is calibrated and validated from known samples using HPLC-DAD. For example, a model equation can be calibrated using multiple spectra of flavonol samples. In some embodiments, the calibration is performed in R software using a PLS package. Two data frames were constructed, the first, having as many rows as samples used in the calibration (64) and as many columns as predictor variables (800 nm-220 nm=580 variables; 550 variables after applying a Savitzky-Golay filter). The values of the data frame being the absorbance of each sample at each wavelength in the spectrophotometer. The second data frame, has as many rows as samples used in the calibration (64) and as many columns as independent variables (3 variables: concentration of 4′OH, 3′4′OH and 3′4′5′OH flavonols). The values of this data frame are the concentration of each of the 3 flavonol groups determined by HPLC-DAD in the solutions used to measure the spectra in the first data frame. A random selection of 68 samples were used to fit the model and the 14 independent samples left were used to validate the performance of the model. This procedure was repeated 100 times yielding determination coefficients ±SD of R²=0.61±0.18, 0.57±0.19 and 0.68±0.16 for the percentage of 4′OH, 3′4′OH and 3′4′5′OH flavonols, respectively.

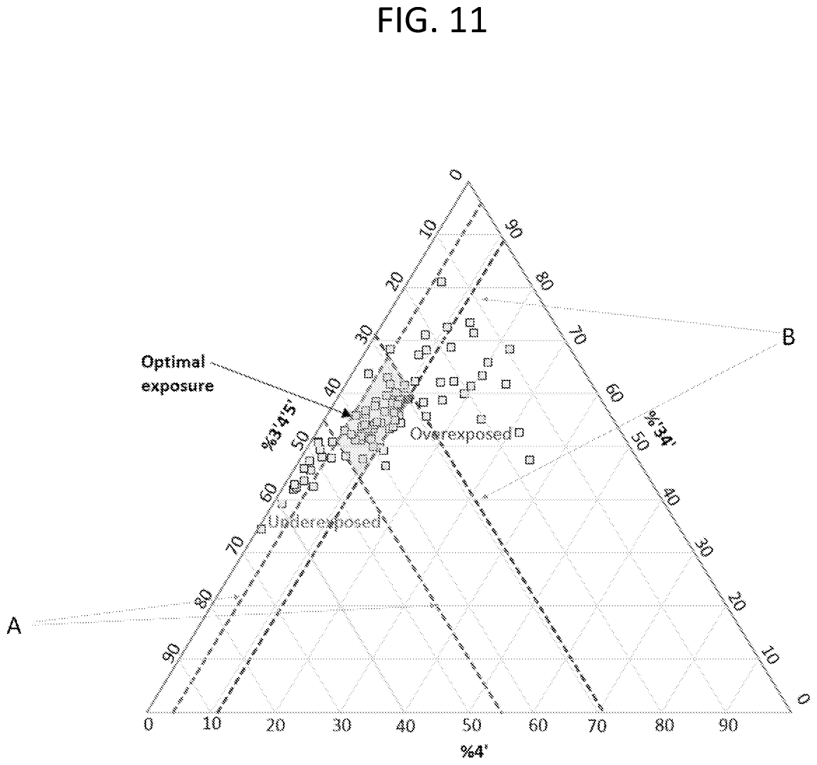

In some embodiments, the PLS model calculates percentage of total flavonol and plots the percentages in a ternary plot for the comparison to grapes with different exposures. See, e.g., FIG. 11.

Once the 4′-OH flavonol, 3′4′-OH flavonol, and 3′4′5′-OH flavonols content percentage of the sample(s) have been determined, they can be compared to one or more threshold value(s) to determine the level of solar exposure of the plant. The inventors have discovered that useful thresholds include, for example:

Using the percentage of 4′-OH flavonol,
if the percentage of 4′-OH flavonol content is below 4%, then the grapes are underexposed and canopy will need pruning, for example in the following season(s); or
if the percentage of 4′-OH flavonol is above 10.5%, then the grapes are overexposed and need cover.
   To further improve accuracy of prediction one can additionally or alternatively use the percentage of 3′4′5′-OH flavonol as follows:
if 3′4′5′-OH flavonol content is above 45%, then the grapes are underexposed and canopy will need pruning, again for example in the following season(s); or
if the percentage of 3′4′5′-OH flavonol is below 30%, then the grapes are overexposed and need cover.

In some embodiments, a computer-based analysis program is used to translate the raw data generated by the detection methods described herein (e.g., the spectrophotometric measurements) into a recommendation for pruning, harvesting, or covering the plant canopy as discussed above.

Any of the analytical steps (converting spectra to flavonol content values or comparing such values to thresholds, or indicating a canopy management step (prune, harvest, cover, etc.) described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Any of the steps of any of the methods can be performed with modules, circuits, or other means for performing these steps. In some embodiments, a spreadsheet (e.g., Excel™) is used for the regression model.

In some aspects of the present disclosure, a computer product is provided. The computer product can comprise a non-transitory computer readable medium storing a plurality of instructions that when executed determine grape canopy management using the criteria described herein.

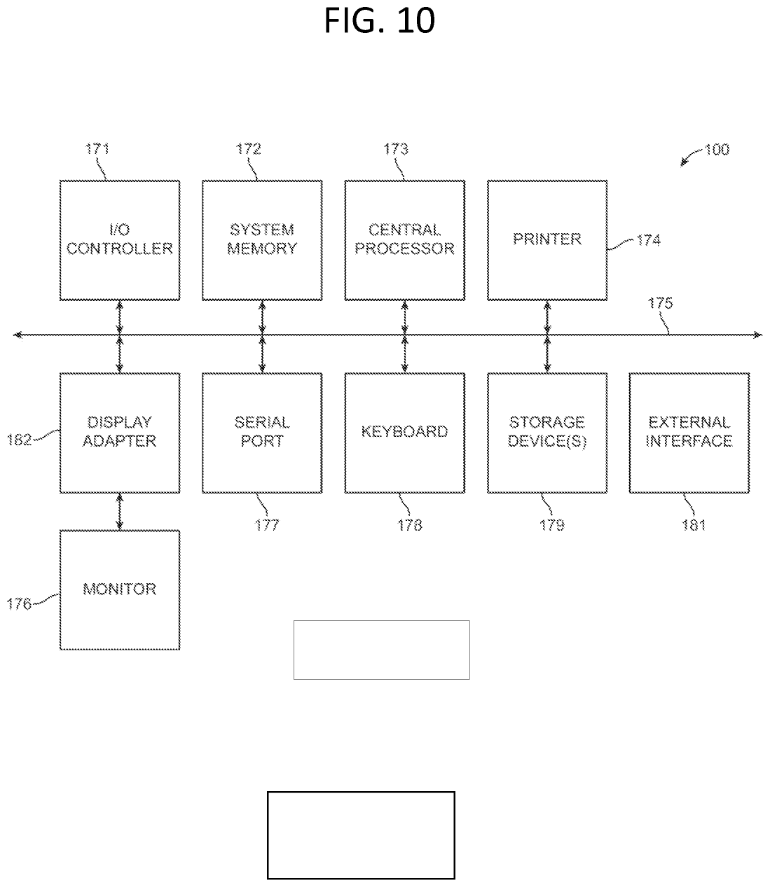

Any of the computer systems mentioned herein may utilize any suitable number of subsystems. Examples of such subsystems are shown in FIG. 10 in computer apparatus 100. In some embodiments, a computer system includes a single computer apparatus, where the subsystems can be the components of the computer apparatus. In other embodiments, a computer system can include multiple computer apparatuses, each being a subsystem, with internal components.

The subsystems shown in FIG. 1 are interconnected via a system bus 175. Additional subsystems such as a printer 174, keyboard 178, storage device(s) 179, monitor 176, which is coupled to display adapter 182, and others are shown. Peripherals and input/output (I/O) devices, which couple to I/O controller 171, can be connected to the computer system by any number of means known in the art, such as serial port 177. For example, serial port 177 or external interface 181 (e.g. Ethernet, Wi-Fi, etc.) can be used to connect computer system 100 to a wide area network such as the Internet, a mouse input device, or a scanner. The interconnection via system bus 175 allows the central processor 173 to communicate with each subsystem and to control the execution of instructions from system memory 172 or the storage device(s) 179 (e.g., a fixed disk, such as a hard drive or optical disk), as well as the exchange of information between subsystems. The system memory 172 and/or the storage device(s) 179 may embody a computer readable medium. Any of the data mentioned herein can be output from one component to another component and can be output to the user.

A computer system can include a plurality of the same components or subsystems, e.g., connected together by external interface 181 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g. an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### V. Grape Canopy Pruning and Harvest

As noted above, the methods described above indicates to a user how to manage the grape plant canopy for optimal grape fruit maturation. When flavonol content indicates the plant has not received adequate solar radiation, the canopy can be pruned to allow additional light to access the grape fruit. For example, leaves, stems or both can be removed from the grape canopy. Harvesting can be achieved by any appropriate method. The amount of pruning can be adjusted depending on the amount of solar radiation already received and based on, for example, desired harvest date.

Alternatively, if the flavonol content indicates the plant has received sufficient solar radiation, then the user may choose to harvest the grape fruit (e.g., without pruning) or cover the canopy with a material that provides partial or complete shade to the grape plant, thereby slowing further ripening of the fruit.

### EXAMPLES

### Introduction to the Examples

Effects of Solar Radiation and Berry Development on Flavonol Profile

In previous research, MyBF1 and FLS were upregulated by UV-B radiation in grapevine (cv. Tempranillo) fruit-bearing cuttings, and grape total flavonol content was linearly related with the time of exposure to an artificial source of UV-B radiation. In the study, the increase in flavonol content was confirmed with cv. Cabernet Sauvignon under field conditions up to ripeness. V vinifera grapes typically go through a ripening process where there is a change in color (degradation of chlorophyll and synthesis of anthocyanins), decrease in astringency (decrease in tannin activity related to proanthocyanin degradation), decrease in acidity (break down of organic acids), and sweetening (accumulation of glucose and fructose mainly). During ripening, flavonol as well as anthocyanin accumulation typically increases sharply up to a point (ca. 23 Brix, ripeness). After that point grape growers leave the fruit in the vine for a variable period (hang time), awaiting for a specific flavor and mouthfeel to develop. Although it is considered that sugar translocation into the berries is no longer taking place during hang time, their content in the pulp may increase due to water loss related to tissue senescence (Keller, 2010). During hang time, flavonoids can degrade even faster than water loss, resulting in decreased concentration, and this decrease may be stronger under high sun exposure (Martínez-Lüscher et al., 2017).

In this study, a period of accumulation during ripening and degradation during hang time was observed. While these two processes took place, flavonol profile kept evolving. Flavonol profile was not significantly different between shading factors before the onset of ripening. However, throughout ripening, 40% shading factor had a stronger increase in the proportion of 3′4′5′-OH flavonols. In addition, whereas the proportion of 3′4′-OH and 3′4′5′-OH flavonols kept evolving even passed ripeness, the proportion if 4′-OH flavonols stabilized and remained unchanged for a period of several weeks.

The process leading to flavonol profile constitution under water deficit and solar exposure has been elucidated (Martínez-Lüscher et al., 2014a). The evolution of flavonol profiles through hang time has not been studied before. One plausible reason for profile changing beyond ripeness, when accumulation balance is negative, and synthesis is no longer taking place, is a differential degradation of the flavonols with different levels of hydroxylation. The addition of substituents of the flavonoid B-ring may strongly increase the antioxidant capacity of the compounds (Arroyo-Currás et al., 2016; Tabart et al., 2009). This higher antioxidant capacity results into higher stability, which could explain the differential degradation observed in this study. It is also noteworthy that F3′5′H has been reported to have affinity, not only for dyhidroflavonols, but also flavonols (Kaltenbach et al., 1999). In fact, Kaltenbach et al. (1999) reported that, in vitro, 3′4′-OH substrates may be easily taken by F3′5′H when 4′-OH substrates are depleted. Therefore, although metabolic channeling could avoid this (Winkel, 2004), an active conversion of 3′4′-OH flavonols into 3′4′5′-OH flavonols after ripeness could not be ruled out. There are many uncertainties around how the diversification of flavonoids takes place in vivo beyond levels of expression of genes encoding for their synthesis enzymes, substrate affinities of these, and differential degradation. To what is known, enzymes of the phenylpropanoid pathway have a spatial arrangement in the cell, using channeling between enzymes, which allows the sequestration of toxic intermediates and controlling flux among branch pathways (Winkel, 2004).

Effects of Canopy Size and Water Status on Flavonol Content and Profile

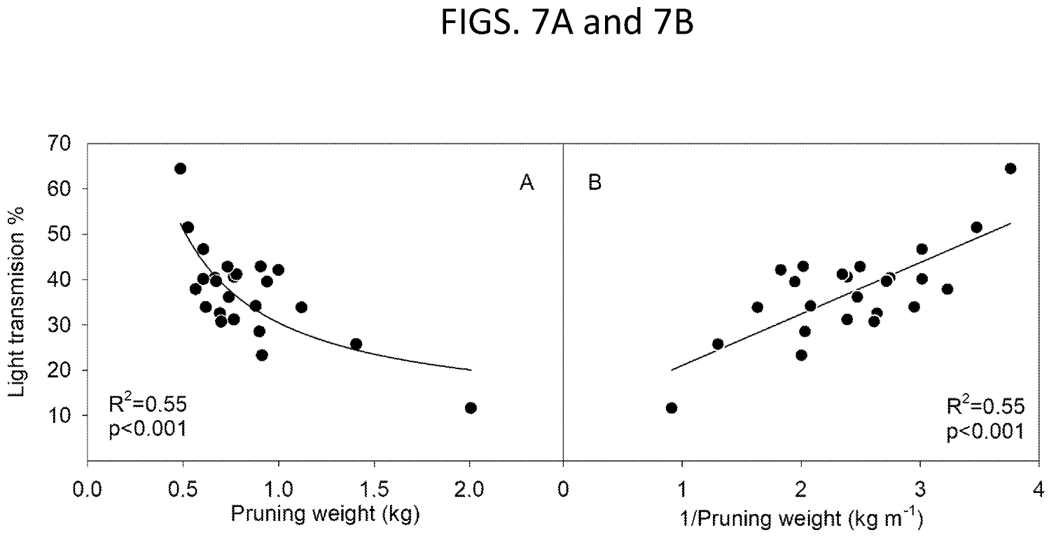

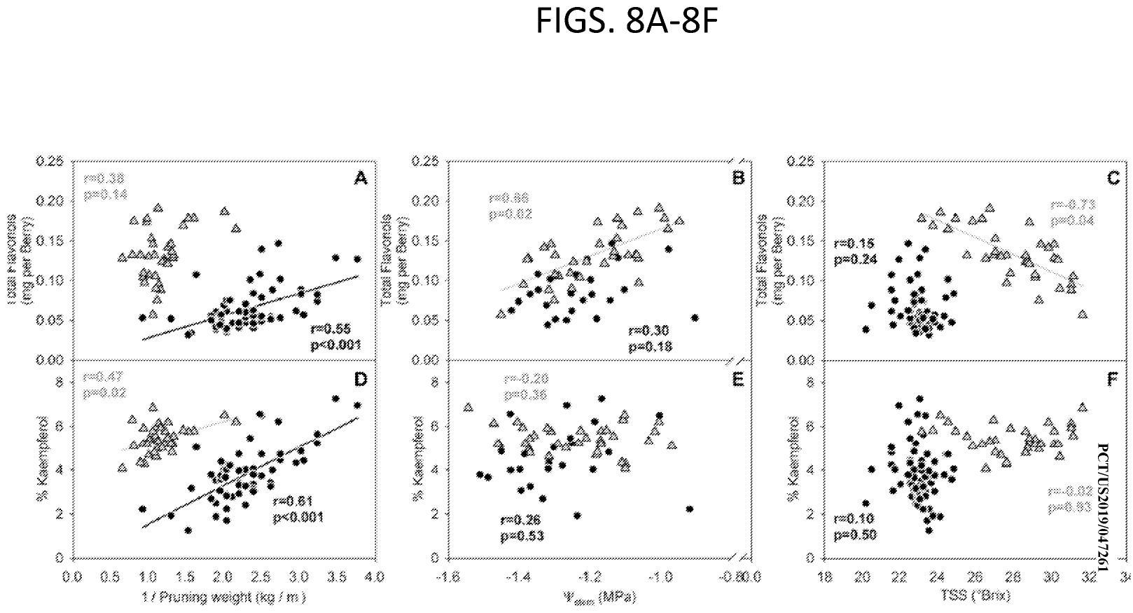

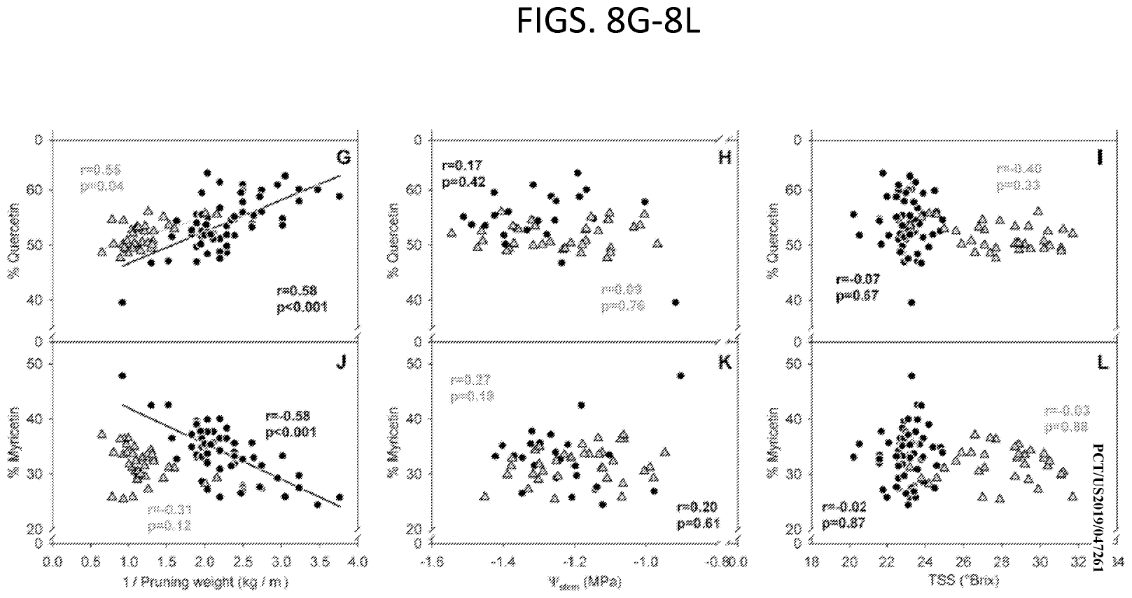

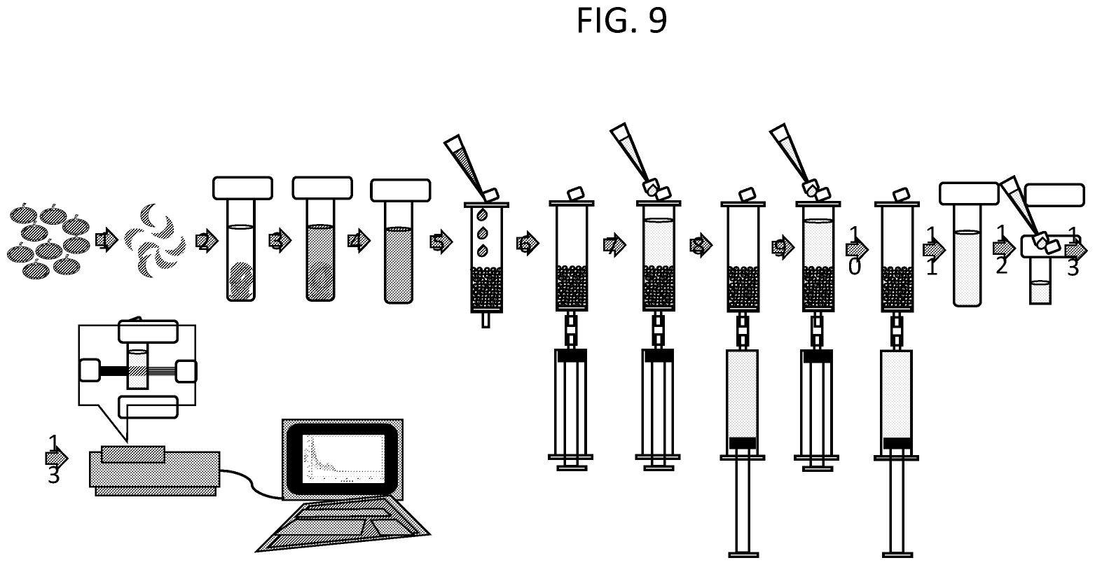

Pruning weights are collected in as a standard practice in viticulture research given its reliability as a proxy of the photosynthetic biomass. The ratio of yield divided by pruning weight (i.e., Ravaz index) is used as an indicator of the balance between sinks and sources, which promotes an adequate ripening (Kliewer and Dokoozlian, 2005). In addition, in training systems where there is a localized fruit zone under the canopy, pruning weights are related to the extinction of light trough the canopy (FIGS. 7A and 7B), and in turn, related to flavonol profiles (FIGS. 8A-8L). However, other systems, for instance divided canopy trellis, where vegetation is compelled up and downwards to expose fruit through a gap in between, pruning weights are not necessarily an estimator of light extinction at the fruit zone.

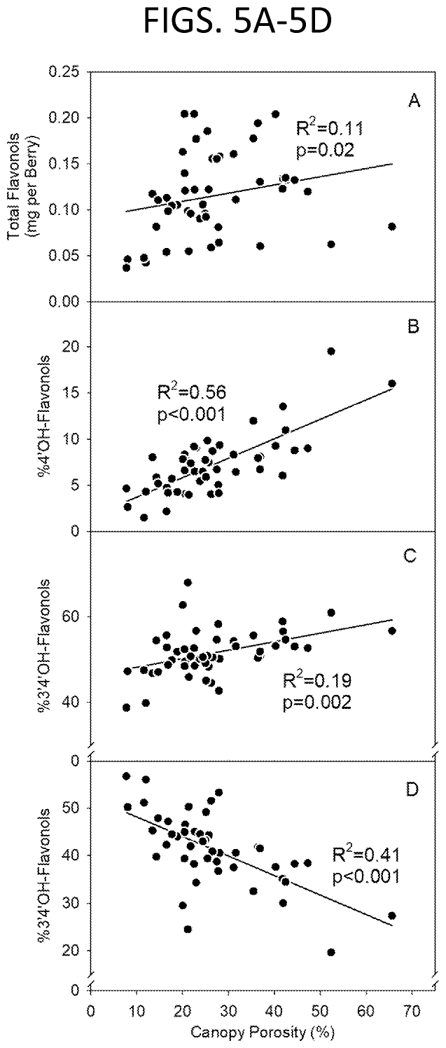

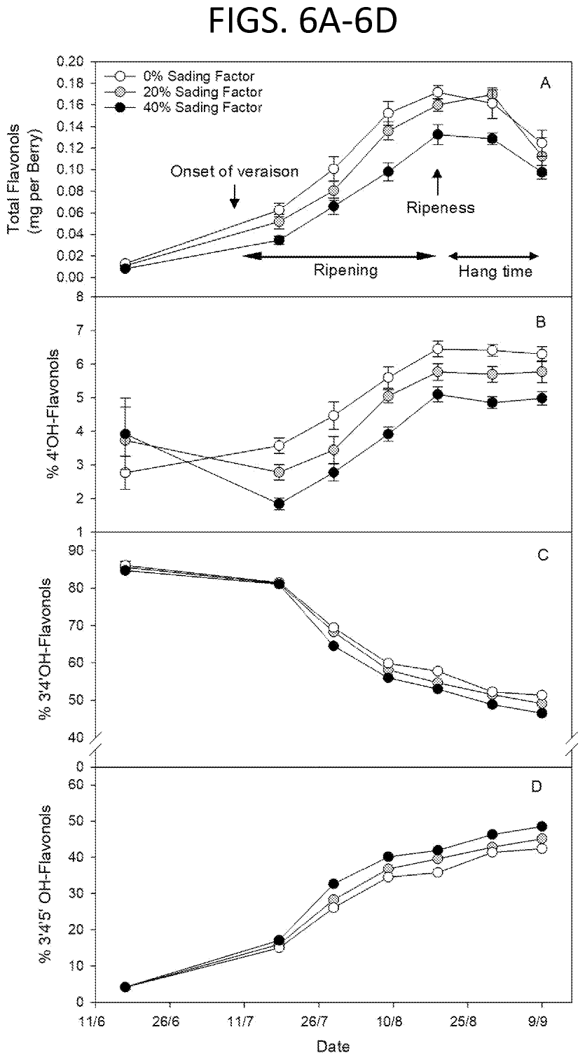

In previous research, flavonol profiles were shaped through the interaction of UV-B radiation and water deficit (Martínez-Lüscher et al., 2014a). Although the proportion of 4′, 3′4′, and 3′4′5′-OH were greatly affected by UV-B, the proportion 3′4′ and 3′4′5′-OH flavonols was also affected to some extent by water status (Martínez-Lüscher et al., 2014a). In the data analysed by this study, mean stem water potentials had a wide range of variation (−1.42 to −0.90 MPa) and still the correlation between stem water potentials and the proportion 3′4′ and 3′4′5′-OH flavonols was not significant. One reason for the lack of effect of water status may be the overriding effect of solar radiation on flavonol profiles. Another explanation for this lack of effect is that the datasets used had a significant range of variation in ripening (i.e., TSS). As discussed before, flavonol profiles are highly sensitive to the progress of ripening (FIGS. 5C and 5D; FIGS. 8A-8L).

Using the Percentage of 4′-OH Flavonols as Metabolic “Data Logger” of Solar Radiation Incidence on Each Individual Grapes

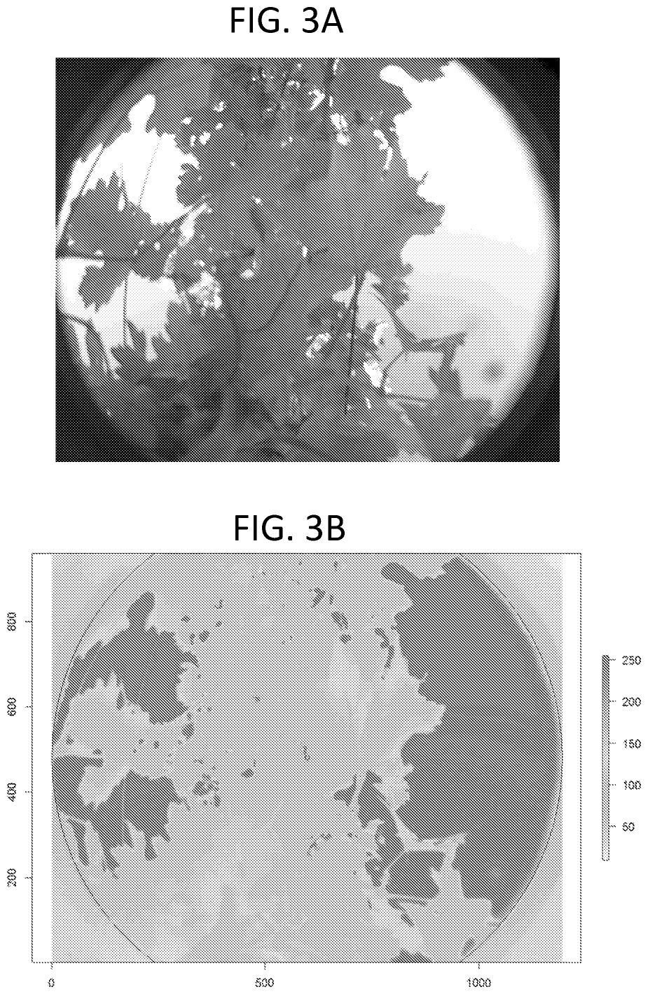

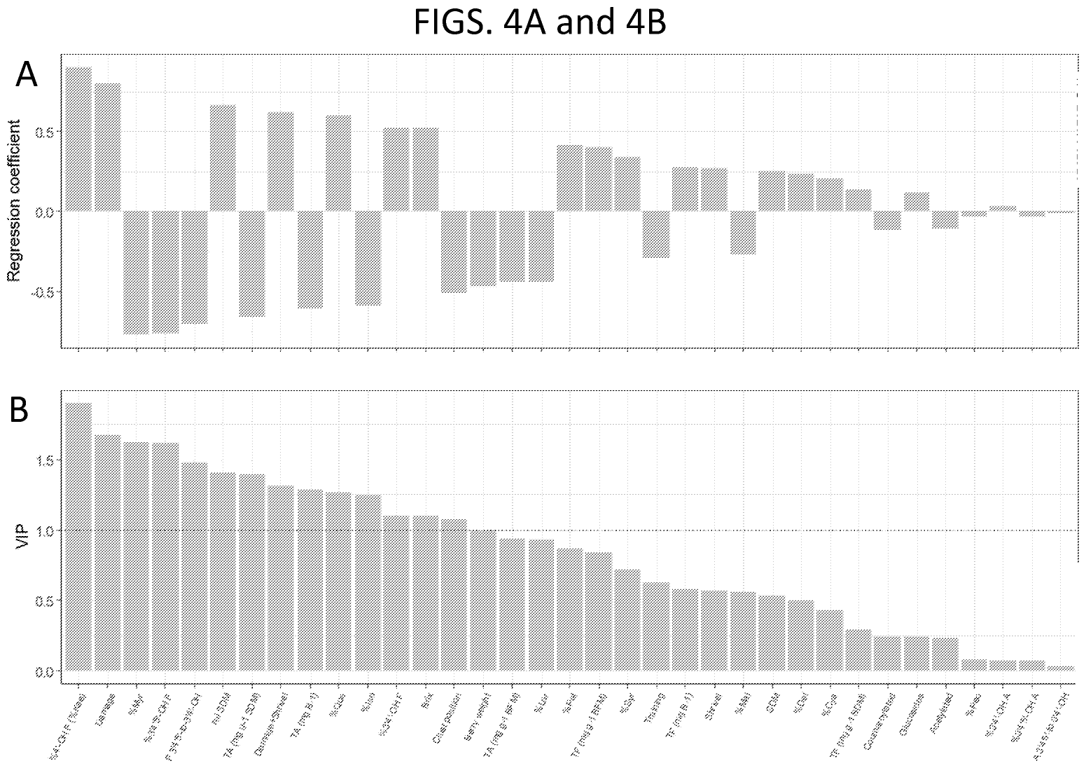

The percentage of 4′-OH flavonols was highlighted as a great predictor of solar exposure and they had a strong linear correlation (FIGS. 3 and 4B). This indicator performed better than other flavonol ratios and even visual sun damage evaluation. It has been pointed out also in previous research that total flavonol concentration of some flavonols and the proportion of 3′4′-OH and 3′4′5′-OH flavonols had a strong correlation to exposure to UV-B radiation (Martínez-Lüscher et al., 2013a). However, concentrations may be susceptible to net synthesis but also degradation in response to solar radiation (Martínez-Lüscher et al., 2017) and the proportion of 3′4′-OH and 3′4′5′-OH may be affected by the progress of ripening or water availability (Martínez-Lüscher et al., 2014a). The results opened the possibility of using flavonol profile for accounting for mean radiation received by a specific berry or the berries in a cluster, a vine, or a vineyard. A great number of studies have been aiming to assess the effect of solar exposure on, for instance, the aroma composition (Koch et al., 2012; Ryona et al., 2008), organic acids (Reshef et al., 2017), anthocyanins (Chorti et al., 2010; Tarara et al., 2008), and proanthocyanins (Cohen et al., 2012). For the study of the effects of solar radiation on grapes, clusters were placed in boxes (Cortell and Kennedy, 2006; Downey, 2004), covered by shade nets (Koch et al., 2012; Reshef et al., 2017), illuminated with lamps (Martínez-Lüscher et al., 2014b), exposed trough leaf removal (Matus et al., 2009; Pastore et al., 2013), or simply natural variation in cluster exposure was captured with a quantum sensor (Bergqvist et al., 2001). The use of the proportion of 4′-OH flavonol as a record of overall grape exposure allows to study the effect of different levels of solar radiation as a continuous variable, in opposition to study it in an ordinal variable (i.e., in treatments), to fine-tune the optimal level of exposure to achieve the most desirable grape composition.

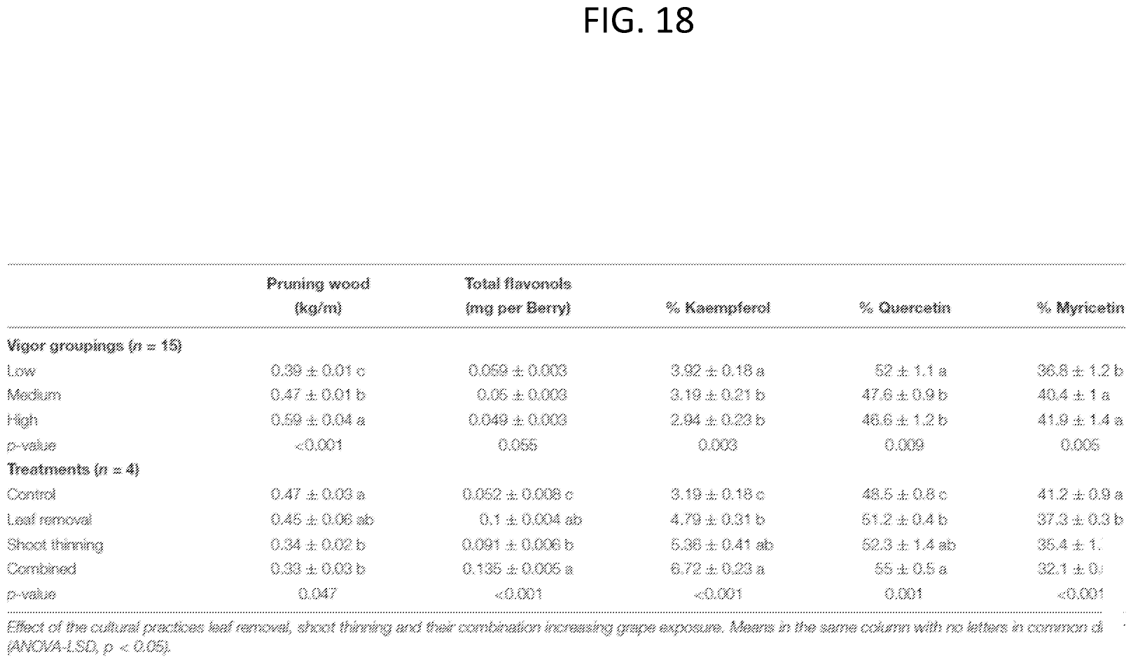

Other metabolic biomarkers are already widely used in research and are starting to permeate into commercial practices. For example, C13 was first reported by Wickman (1952) and in grape juice by Gaudillere et al. (2002). C13, a stable isotope signal in plant tissues in general, but especially in C3, is an integrator of photosynthetic performance mediated by stomatal conductance. In brief, there is a lower diffusivity of C13 through the stomatal pathway and a fractionation by RUBISCO related to sub stomatal CO₂ levels (Farquhar et al., 1989), which makes plants grown under water deficit have a lower proportion of C13 in their carbon pool. Given the importance of maintaining an adequate level of water stress in the production of quality red wine grapes, there is a need for verification of the cultural practices used throughout the season. The analyses of stable isotopes is an affordable option for this purpose, and it has been widely used in studies as a verification for plant water status (de Souza et al., 2003; Gibberd et al., 2001; Koundouras et al., 2008). In a similar way, there is a need for verification of light exposure in relation to canopy management practices. The correlation of 4′-OH flavonol with solar exposure could be useful to verify the changes in solar radiation reaching the fruit in response to treatments aiming to alter fruit microclimate. In the results, a vineyard with presumably an excess of vigor, the amount of solar radiation reaching the grapes needed to be adjusted to reach an optimal grape composition (Haselgrove et al., 2000; Koch et al., 2012). Leaf removal and shoot removal are the most widely used cultural practices for this purpose, the second being more effective in increasing light transmission (FIG. 18) but at a cost of yield reduction. With treatments of leaf removal, shoot removal, and their combination, we achieved increasing proportions of 4′-OH flavonols due to a higher exposure to solar radiation. However, it is noteworthy that the increase in the solar radiation received by the grapes was not significantly different between leaf removal and shoot removal. This suggests that in this case, removing the first 4 leaves from every shoot on the north side may have similar results as a 30% removal of the canopy through shoot removal.

Currently, the ways to estimate solar radiation transmission into the fruit zone are sensors that allow integrating radiation readings either through space (i.e., line quantum sensor) or time (i.e., data logger), but not both, as the determination of the proportion of 4′-OH flavonols of a representative sample of grapes. Although flavonol profiles may be an arbitrary scale, it can be calibrated against values of accumulated radiation (Martínez-Lüscher et al., 2014b). Given the robustness of the response of the proportion 4′-OH flavonols to solar radiation, the calibration of this biomarker for a given genotype should not be influenced by crucial factors in viticulture such as hang time or water deficit, allowing comparisons between different years and plantings.

### Example 1—Materials and Methods

Plant Water Status

Plant water status was assessed by means of stem water potential (Ψstem) measured around solar noon (12-14:00). Three leaves from each experimental unit were covered with a zip-top plastic bag and aluminium foil for two hours. Leaves were exscinded with a razor blade and Ψstem was determined using a pressure bomb (model 615, PMS).

Canopy Size

Canopy size was estimated by dormant pruning weights. Although pruning weights only include non-photosynthetic tissues and not leaves biomass which most certainly is the most determinant factor, it is a quick and reliable indicator of canopy size used in viticulture. After leaf fall, last year's growth was exscinded from each plant with secateurs and individually weighed. Normalized vegetation difference index (NDVI) was taken using a Crop circle ACS-430 sensor connected to a GPS Geoscout X (Holland Scientific, Lincoln, USA) and a GPS antenna GPS18x (Garmin, Switzerland) mounted onto an all-terrain vehicle; passing through each row. The average of the NDVI recorded through each location was associated to each experimental unit.

Statistical Analyses

Partial least squares regression (PLSR) with a single-response orthogonal scores algorithm was used to evaluate the strength of berry composition and traits as predictor variables of canopy porosity using ‘pls’ package (version 2.6-0) for R programing language (version 3.2.5-6, R core). PLSR model was constructed with two latent factors for all the predictor variables (cluster location, berry traits and composition) and the dependent variable (canopy porosity). PLSR, contrarily to other regression methods, can analyse the effect of a large number of predictor variables (compared to the number of observations) with a high degree of autocorrelation among them, as is the case. PLSR gives two major outputs for each predictor variable: variable importance in the projection (VIP) and model coefficient. VIP is related to the weight on a particular variable used to fit the PLS model. Values above 0.8 are considered to indicate a significant importance of the variable (Wold et al., 2001). Model coefficient, which can be either positive or negative, indicates a positive relationship between variation in the predictor variable and the dependent variable in the case of positive coefficient and a negative relationship in the case of negative relationship.

Linear and non-linear regression was performed with Sigmaplot (13.0) applying a second order function if needed. Correlation was analysed using Spearman rank test. For data arranged by categorical factors (treatments), ANOVA combined with a LSD post hoc was used (R package ‘agricolae’ version 1.2-8).

### Example 2—Characterization of Individual Clusters

Four rows of plants (Cabernet Sauvignon Foundation Plant Services clone 7 grafted onto 420A) were pruned, either spur pruned compelling vegetation upwards (vertical shoot positioned; VSP) or cane pruned leaving 50 cm canes and free vegetation (Sprawl). Within these rows, 48 Clusters from 8 plants (4 each of the training systems) were flagged. Pictures from the cluster perspective pointing at the sky were taken using 150° fish-eye lens. These pictures were processed in R (version 3.2.5-6) using package raster (version 2.6). Pictures were cropped with a circle and channel Blue was used to discriminate the pixels capturing the sky or clouds from those capturing the canopy or trellis. Porosity was calculated as the percentage of sky/clouds pixels in the cropped picture. When ripeness was reached (>22° Brix), sun damage and shriveling was visually recorded in a scale from 0 to 3, and 5-berry samples were collected from the top of each cluster. Grapes were weighed and skinned. TSS were determined from the pulps juice and skins were collected and stored at −80° C. for later analyses of anthocyanins and flavonols through HPLC-DAD.

### Example 3—Evolution of Grapes Under Different Shading Factors

An experiment was conducted in 2016 in a premium vineyard in Oakville, Calif. (38.428° N, 122.409° W). Plants were 7-year-old Cabernet Sauvignon (clone 7) grafted into 110R with a bilateral double cordon trellis with vegetation compelled upwards and a vine spacing of 2.4 by 2 m. A control (uncovered) and two shade nets treatments (20% shade factor and 40% shade factor polyethylene; Ginegar, Kibbutz, Israel) were installed on 27 May 2016 (31 days after anthesis). The uniformity of the nets light transmittance throughout the solar spectrum was verified with a spectrometer (Black Comet-SR, StellarNet; Tampa, Fla., USA). In the range of 300 to 1000 nm, the minimal transmittance from was 77% and 58%, the maximal was 84% and 61%, and the average 81% and 60% for the 20% and 40% shading factor respectively. On dates 20 Jun., 19 Jul., 29 Jul., 9 Aug., 19 Aug., 29 Aug., and 9 Sep. of 2016, 20-berry samples from each experimental unit were collected weighed and stored at −80° C. for later analyses of anthocyanins and flavonols through HPLC-DAD.

### Example 4—Natural and Induced Variability of Commercial Vineyards

Two commercial vineyards were followed during 2016 season across central California (i) Healdsburg (38.66° N; 122.91° W), a 19-year-old Cabernet Sauvignon grafted into 110R (V. berlanderi Planch×V. rupestris Scheele) with quadrilateral (H-shaped) cordon trellis and a vine spacing of 2.14×3.35 m (vine×row); and (ii) Paso Robles (35.58° N 120.63° W), a 14-year-old Merlot grafted into 1103P (V. berlanderi Planch×V. rupestris Scheele) with double cordon trellis VSP and a vine spacing of 1.83×2.44 m (vine×row). Within these vineyards, experimental units consisting in 5 plants each were spread through each plot in a grid. In Paso Robles vineyard, in order to emulate the most common industry practices, leaf removal (North east side), shoot removal (down to 13.6 per meter), and leaf and shoot removal was applied randomly on 4 experimental units each July 27th to 12 randomly distributed experimental units. When commercial maturity was reached, 20-berry samples for each experimental unit were collected, weight and stored at −80° C. for later analyses of anthocyanins and flavonols through HPLC-DAD.

### Example 5—Berry Skin Analyses

Berries were gently skinned and skins were freeze-dried (Cold Trap 7385020, Labconco, Kansas City, Mo., USA). Dried tissues were ground with a tissue lyser (MM400, Retsch, Germany). Fifty mg of that powder were extracted with methanol:water:7M hydrochloric acid (70:29:1, V:V:V) to determine the concentration anthocyanins and flavonols. Extracts were filtered (0.45 μm, Thermo Scientific) and analysed by a HPLC-DAD (1260 Series, Agilent, Santa Clara, Calif.). HPLC method was adapted from previous methods for flavonols that use formic acid as acid and acetonitrile as organic solvent (Downey and Rochfort, 2008; Hilbert et al., 2015). To quantify flavonols, these methods required anthocyanin removal with a solid phase extraction with a cationic exchange resin (Hilbert et al., 2015) or using a column with the same characteristics (Downey and Rochfort, 2008) to avoid co-elution of flavonols and anthocyanins which interferes the quantification of flavonols given the non-negligible absorbance of anthocyanins at 365 nm.

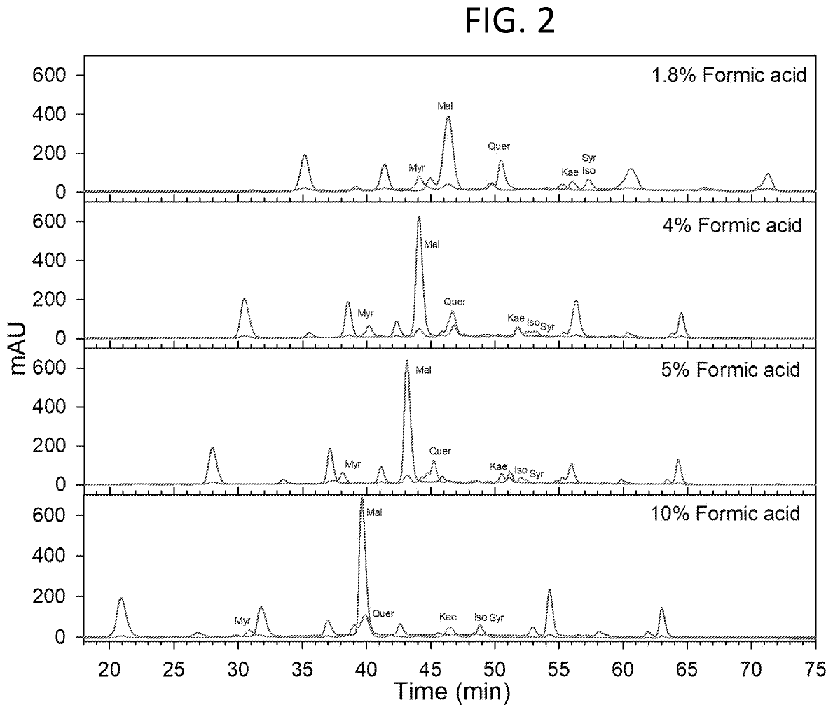

HPLC apparatus was an Agilent 1260 series (Agilent, Santa Clara, USA). Starting from the procedures reposted by Downey and Hilbert et al., (2015) conditions were adapted to a reverse phase C18 column LiChrosphere® 100, 250×4 mm with a 5 μm particle size and a 4 mm guard column of the same material. Flow was set to 0.5 mL to avoid exceeding the columns maximum pressure (110 bars) and temperature was set to 25° C. Although higher temperature can reduce the density of the mobile phase sometimes improving chromatography results, it is noteworthy that some phenolic compounds such as anthocyanins are sensitive to thermal degradation (Sadilova et al., 2007). Two mobile phases were designed to always maintain the following proportions (V/V) of Acetonitrile, 0-8 min 8%, at 25 mins 12.2 T, at 35 mins 16.9, at 70 mins 35.7%, 70-75 mins 65% and 80-90 min 8%. For the fine-tuning of the method for the quantification of flavonols, different isocratic concentrations of formic acid were tested from 1.98 to 10% (FIG. 2). The remaining volume up to 100% was achieved with purified water.

Identification of anthocyanins was performed by comparing retention times with previous methods using mass spectrometry (Martínez-Lüscher et al., 2014b). Anthocyanins were quantified by determining the peak area of the absorbance at 520 nm. Malvidin-3-O-Glucoside (Extrasynthese, Genay, France) was used as a quantitative standard for anthocyanins. For the identification of flavonols, standards of myricetin-3-O-glucoside, quercetin-3-O-galactoside, quercetin-3-O-gluconoide, quercetin-3-O-glucoside, kaempferol-3-O-glucoside, isorhamnetin-3-O-glucoside, and syringetin-3-O-glucoside (Extrasynthese, Genay, France) were used. Flavonols were quantified by determining the peak area of the absorbance at 365 nm. Quercetin-3-O-glucoside was used as a quantitative standard for all the flavonols. It must be noted that each individual anthocyanin or flavonol have a different molar relative response factors (e.g., absorbance per mol) and even though calculating a response factors for each flavonol would have been possible, this is not the standard practice in the literature and would make comparisons of flavonol profiles harder.

### Example 6—HPLC Methods to Avoid Co-Elution

HPLC methods can be fine-tuned to avoid the co-elution of flavonol and other phenolic compounds. The incremental concentrations of formic acid reduced the retention time of all compounds. However, this reduction was differential, especially when comparing anthocyanins and flavonols. For instance, malvidin-3-O-glucoside retention time changed from 46.3 min at 1.8% to 39.6 min at 10%, whereas for quercetin-3-O-glucoside retention time was 50.46 min at 1.8% and 39. min at 10%. This change was rather linear and corresponded to −0.80 and −1.29 minutes (advance) for every 1% increase in formic acid concentration for malvidin-3-O-glucoside and qercetin-3-O-glucside, respectively. This difference results into a shift of flavonols appearing earlier than anthocyanins for increasing formic acid concentrations. In the case of quercetin glucoside and malvidin-3-O-glucoside, this shift was 0.49 minutes for every 1% increase in formic acid concentration in the mobile phases.

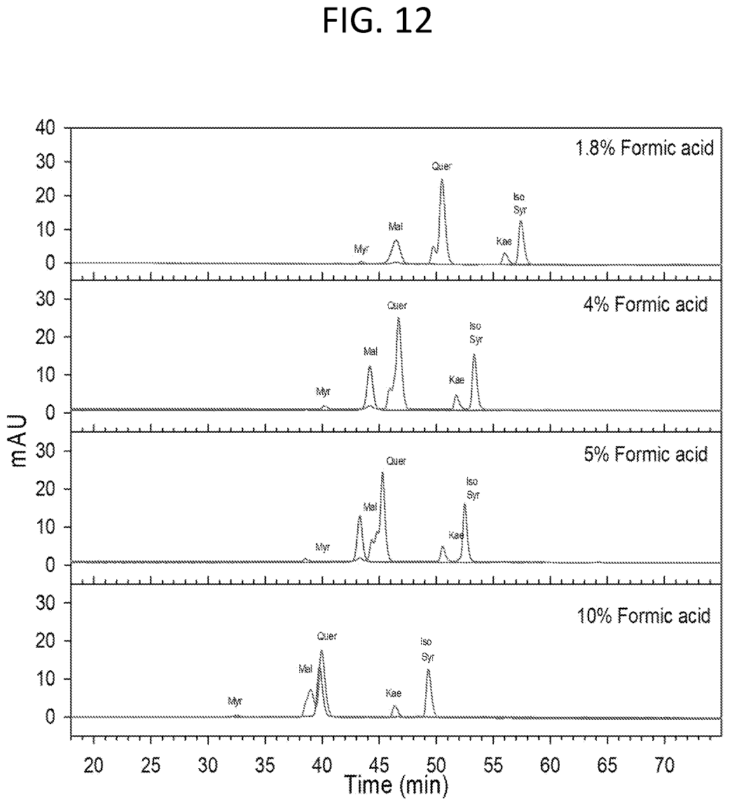

The three quercetin glycosides most frequently found in V vinifera grapes and present in the standard used (FIGS. 2 and 12) were not separated under all conditions. Only a concentration of 6% of formic acid resulted in three peaks. Although a concentration of 10% could have separated the three compounds, these co-eluted with malvidin-3-O-glucoside.

At low concentration of formic acid (1.8%), peak sharpness was lower than under any other condition, reducing peak height down to 390 mAU for malvidin-3-O-glucoside (and 159 mAU for quercetin-3-O-glucoside), compared to the 624, 644, and 690 mAU at concentrations of 4, 6, and 10% of formic acid, respectively.

This reduction of sharpness was associated to lower peak area for anthocyanins. For instance, the peak area of malvidin-3-O-glucoside increased from 385 to 422 and 426 mAU in the commercial standard mixture for 1.8, 4, and 5.5% formic acid, respectively (FIG. 12). This was not the case of flavonols for which the area of the combined three quercetin glycosides (Quercetin-3-O-galactoside, Quercetin-3-O-glucuronide, and Quercetin-3-O-glucoside) was 1028, 1016, and 1021 mAU, which suggest that the quantification of flavonols may not be affected by the percentage of formic acid in the HPLC mobile phases.

### Example 7—Grape Traits, Anthocyanin, and Flavonol Profile as a Predictor of Grape Exposure

When sorting variables according to their VIP values, flavonol profile variables %4′-OH, % Myricetin, %3′4′5′-OH, and the ratio 3′4′5′-to-3′4′-OH came in position 1,3,4, and 5 suggest the intimate relationship of flavonol profiles, rather than concentration, and exposure of grapes to solar radiation. Physical determinations such as the visual assessment of fruit damage and relative skin mass came in second and sixth position, respectively. Other variables such as anthocyanin content (either by mg g-1 berry or mg berry-1), total soluble solids (TSS), or cluster position had high VIP values, whereas the trellis system had a low VIP value.

### Example 8—Effect of Shading Factor on Flavonol Profile Throughout Ripening and Hang Time

Grapes grown under a shading net had lower flavonol content throughout ripening. This difference was reduced after ripening, and in fact, this was not significant at harvest. Changes in flavonol profile displayed different patterns among the three major groups of flavonols. For instance, % 4′-OH flavonols increased throughout development in all shading conditions until 19th of August (20.9° Brix), and remained similar from that moment to harvest. The proportion of 4′-OH flavonols were not significantly different before veraison but during ripening the significant trend 0%>20%>40% shading factor appeared. 3′4′-OH and 3′4′5′-OH flavonols were the majority of flavonols (>92%), and therefore, the increase in the proportion of one, results in the decrease of the other, and vice versa. The proportion of 3′4′-OH flavonols decreased in all the treatments until harvest. Increasing shading factors resulted in a lower proportion of 3′4′-OH flavonols. In contrast, increasing shading factors resulted in higher proportion of 3′4′5′-OH flavonols and these differences increased throughout development until harvest.

### Example 9—Response of Flavonol Content and Profile to Canopy Density Overrides the Effect of Plant Water Status and Total Soluble Solids

The transmission of light through canopies followed an extinction function as pruning heights increased. The inversion of pruning weights resulted into a linear positive relationship with light transmission for the range of variation of the dataset (FIGS. 7A and 7B).

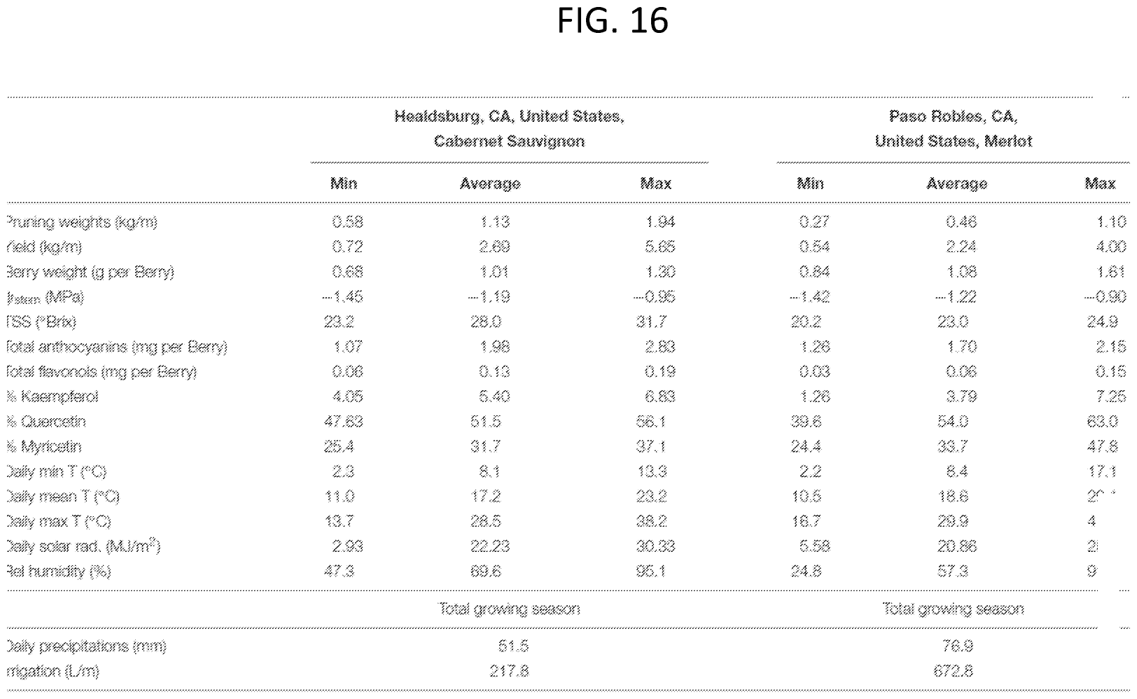

In the two datasets analyzed, the sensitivity of flavonol content and profiles to canopy size, water status, and total soluble solids (TSS) represented two different wine grape production systems (FIG. 16). The two sites, Healdsburg and Paso Robles, had a similar weather conditions in the year of study, with very low precipitations and high temperature throughout the growing season, average conditions for most Californian viticultural regions. Vines were two different varieties on different trellis systems and this most likely affected pruning weights and yields. Cabernet Sauvignon in Healdsburg was higher in the quadrilateral cordon system. These grapes were also much more ripe (23.2-31.7° Brix) than those of Paso Robles Merlot (20.2-24.9° Brix). Inverse pruning weights were strongly correlated to flavonol profile but not the flavonol content (FIGS. 8A-8L). Among these determinations, the proportion of 4′-OH flavonols (kaempferol) had the best coefficients of determination in both Cabernet Sauvignon and Merlot (r=0.47 and r=0.61, respectively). Total flavonol content was strongly correlated to water potentials in Healdsburg Cabernet Sauvignon, suggesting a reduction with increasing water deficit. However, it must be noted that stem water potentials were also strongly correlated with TSS in Cabernet Sauvignon vineyard (r=−0.84; p<0.001; data not shown). Berry TSS only had significant effects on flavonol content and profile in Healdsburg Cabernet Sauvignon. For instance, total flavonol content had a clear decrease with increasing TSS (R2=0.55; p<0.001). The proportion of 3′4′5′-OH flavonols was also significantly reduced with increasing TSS, in favor of 3′4′-OH flavonols.

### Example 10—Response of Flavonol Content and Profile to Canopy Density Overrides the Effect of Plant Water Status and Total Soluble Solids

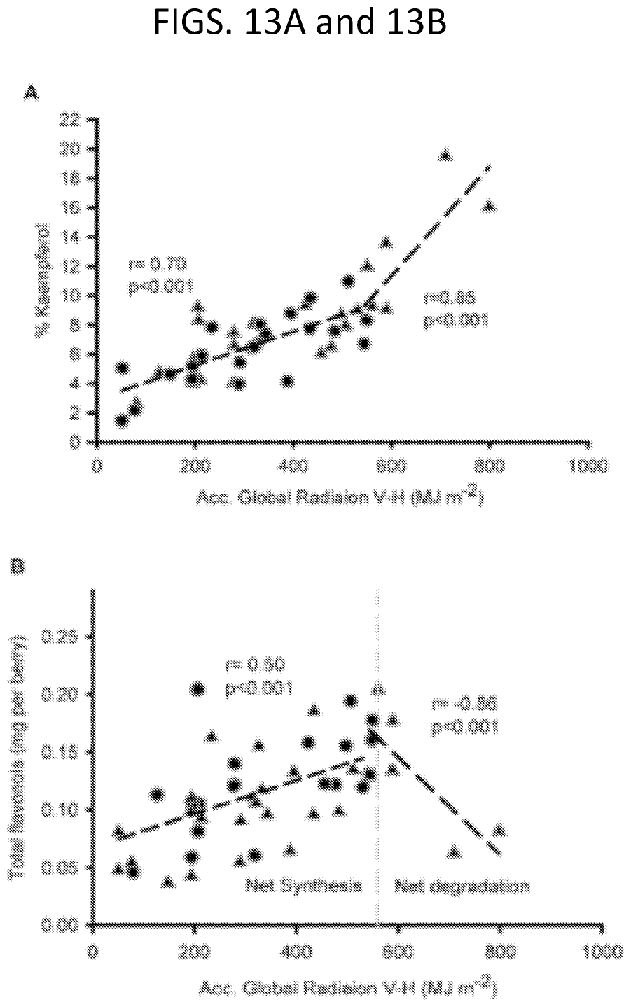

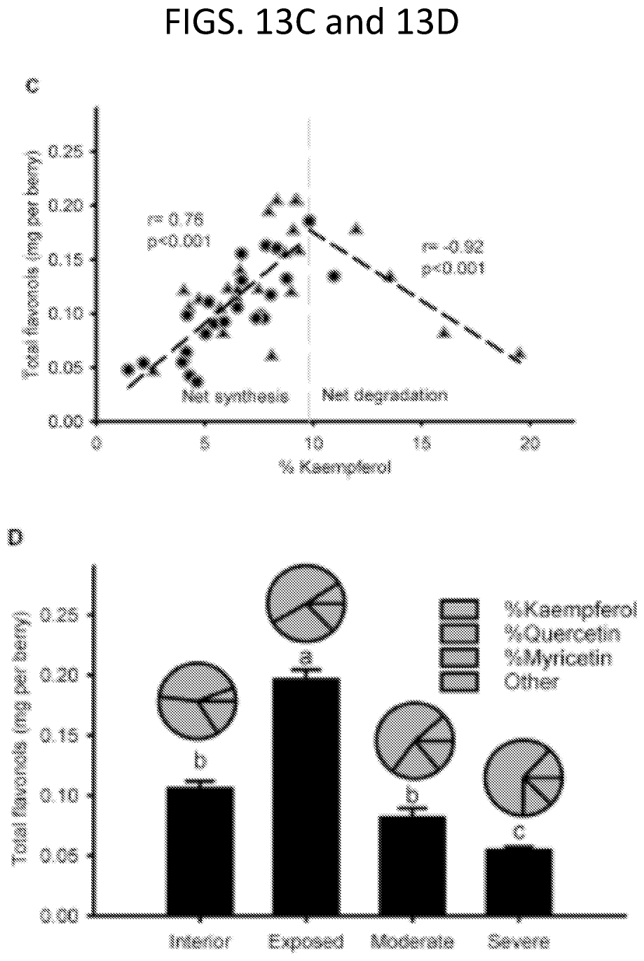

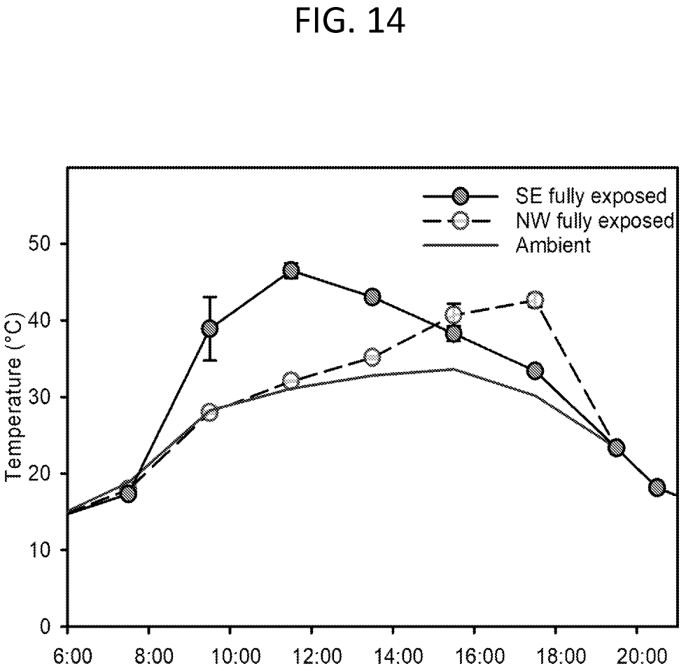

Modeled accumulated radiation was strongly correlated to the percent of kaempferol (FIGS. 13A-13D), with a stronger gradient for higher doses of global radiation. Breaking point for the relationship between global radiation and percent of kaempferol was found at 544.2 MJ m⁻²; similar to the relationship between global radiation and total flavonols, which had a breaking point at 560.2 MJ m⁻². Thus, two phases were visible in the response of flavonols to solar radiation: A first one, where flavonols increased (i.e., net synthesis) up to approximately 550 MJ m⁻² from beginning of ripening to harvest (11.7 MJ m⁻² per day); and a second one, where total flavonols decreased abruptly with doses above 550 MJ m⁻² (FIG. 13B). This relationship was sharper when the percent of kaempferol was used as an indicator of solar radiation received by the grapes (FIG. 13C). The temperature of fully exposed clusters in this vineyard measured on 11 September reached a maximum average temperature of 46.5° C., 15.4° C. above air temperature (FIG. 14). These results were supported by the higher content of flavonols in exposed clusters and the abrupt decrease in those grapes with visual symptoms of overexposure (FIG. 13D). Despite these changes in concentration, flavonol profile only changed in one direction as exposure increased; increasing the percent of kaempferol and the percent of quercetin in detriment to the percent of myricetin. The rest of the flavonol profile was constituted by methylated flavonols that remained constant ca. 18% from interior through overexposed grapes.

### Example 11—Relationship Between Flavonol Content and Profile to Predict with Canopy Porosity and Leaf Area Index (LAI)

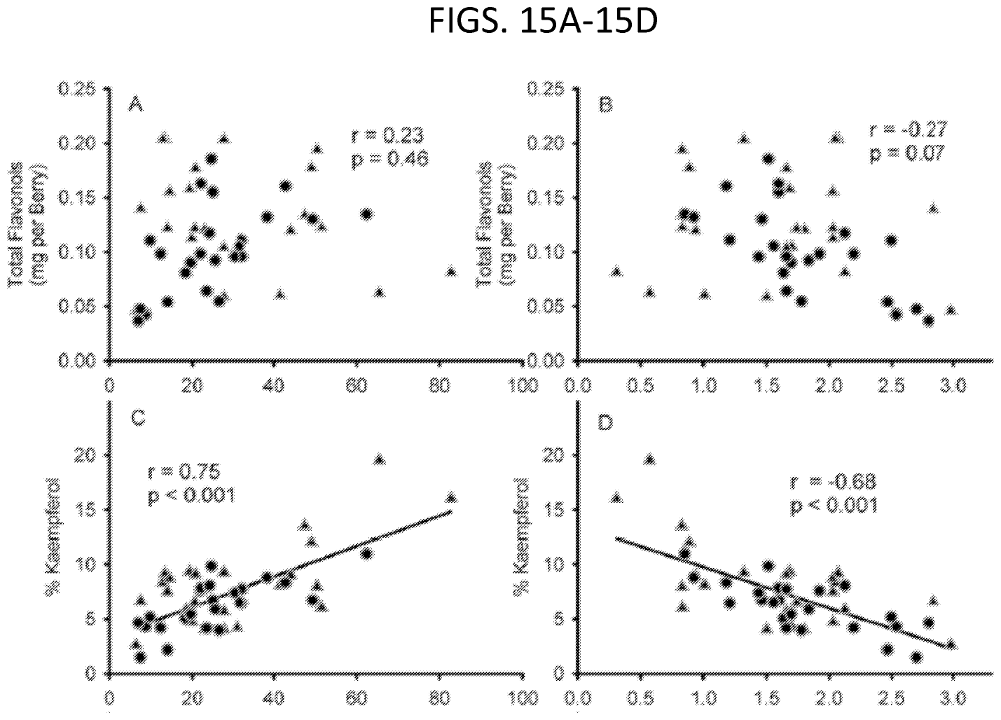

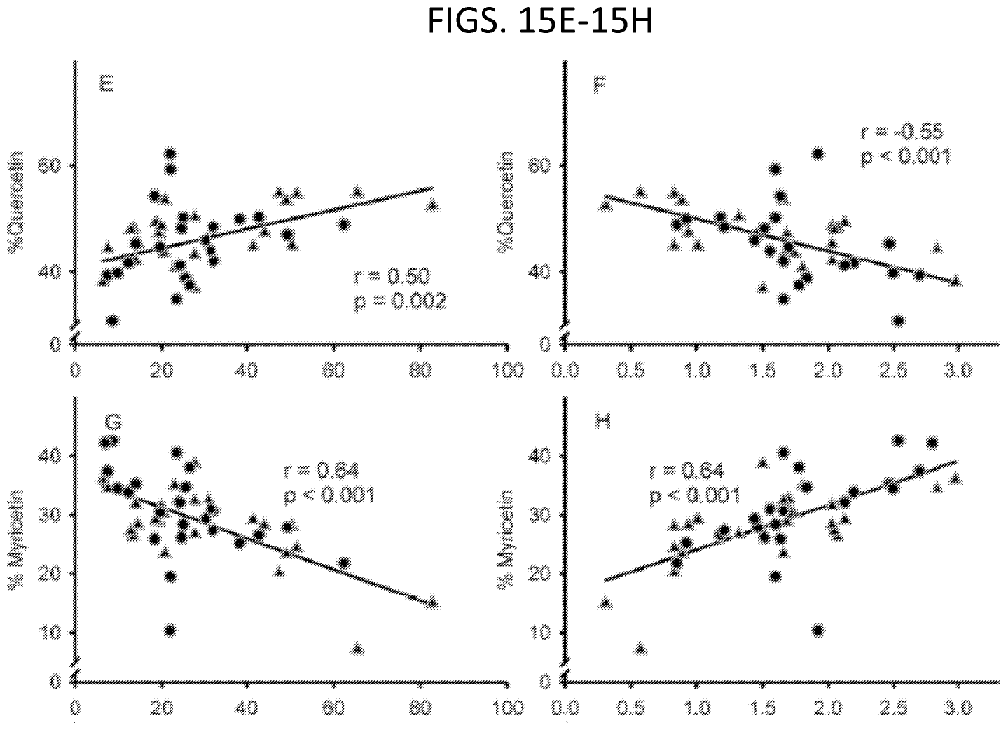

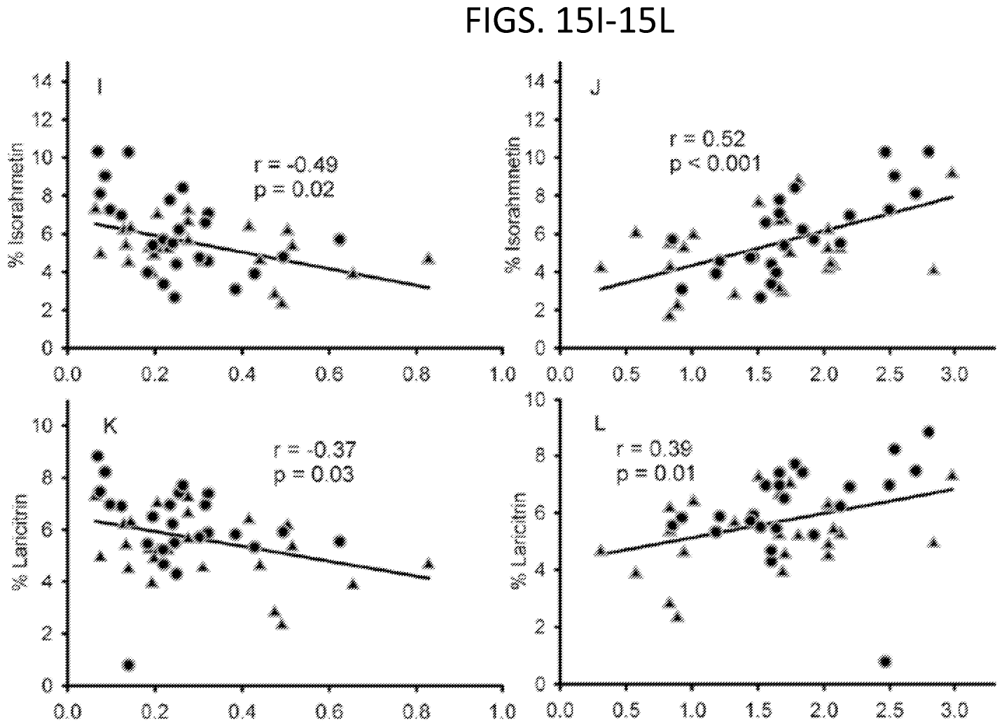

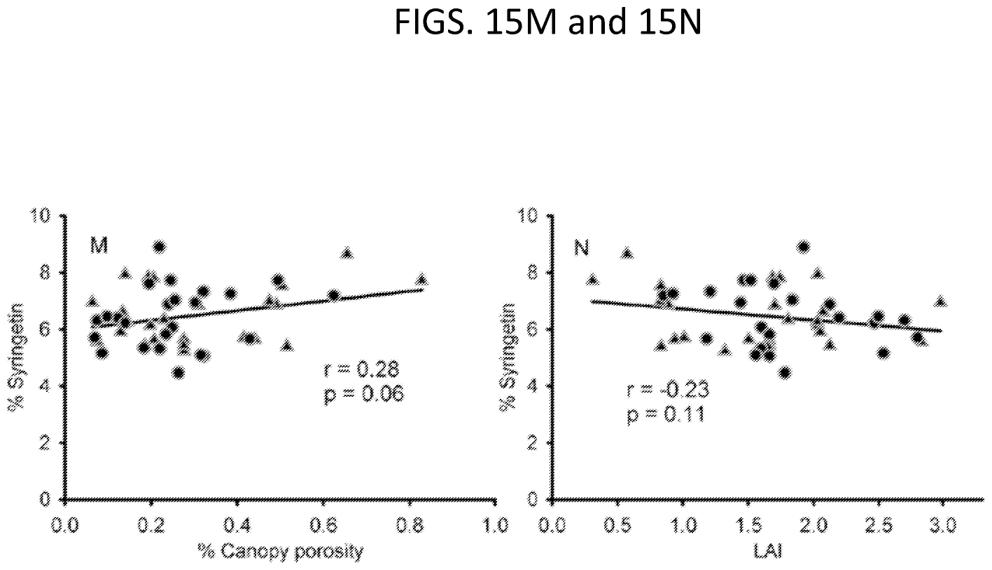

Total flavonols and canopy porosity did not show a significant trend (FIG. 15A). Correlations were stronger for the proportion of kaempferol (r=0.75; p<0.001; FIG. 15C) compared to quercetin (r=0.50; p=0.002; FIG. 15E) and myricetin (r=0.64; p<0.001; FIG. 15G) flavonols. LAI, which had an inversely proportional relationship to canopy porosity (r=0.91; p<0.001; data not shown), did not present a significant correlation with total flavonols, either (FIG. 15B). Consequently, strong correlations were found with the percent of kaempferol (r=−0.68; p<0.001; FIG. 15D), the percent of quercetin (r=−0.55; p<0.001; FIG. 15F) and the percent of myricetin (r=0.64; p<0.001; FIG. 15H). Methylated flavonols had slightly weaker correlations with canopy porosity and LAI and in the case of syringetin these were not significant (r=0.28; p=0.06; FIG. 15M). The percent of laricitin had similar significant correlations with canopy porosity (r=−0.37; p=0.03; FIG. 15K) and LAI (r=0.39; p=0.01; FIG. 15L) as the percent of myricetin. However, in the case of isorhamnetin and syringetin, their relationships with canopy porosity and LAI were the contrary to their non-methylated homologs FIGS. 15I, 15J, 15M, and 15N) (quercetin and myricetin, respectively).

### Example 12—Natural Spatial Variability of Grape Light Interception and Effect of Canopy Management Practices

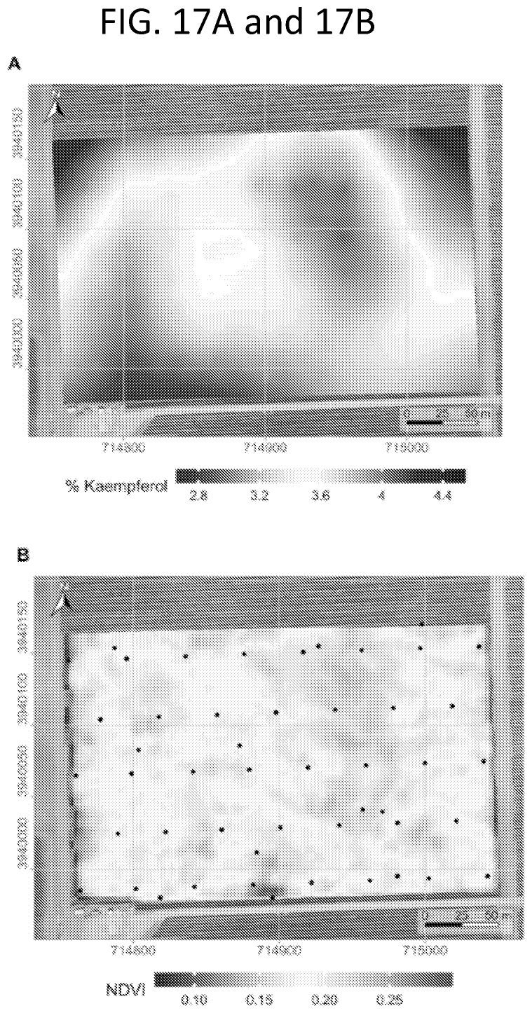

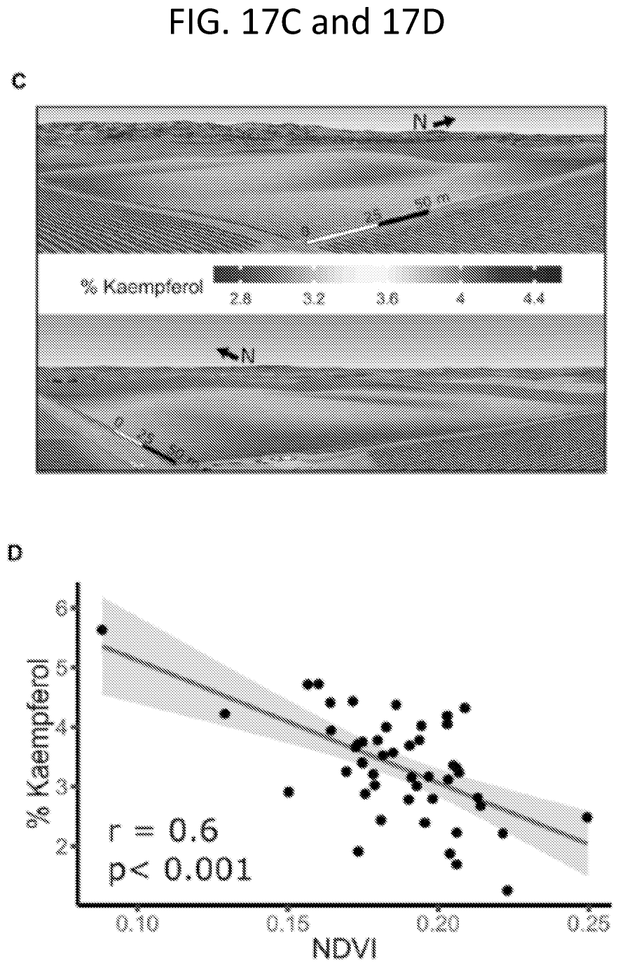

Spatial variability in the proportion of kaempferol in flavonols of Merlot was further studied. Spatial interpolation of the percent of kaempferol displayed a distinct spatial pattern based on semi-variogram investigations (data not shown). Because of the relationship between elevation and the proportion of kaempferol, the latter was kriged using universal block kriging with elevation as a covariate (FIG. 17A in 2D and FIG. 17C in 3D). Including elevation in the geostatistical model improved the results when compared to the ordinary kriging using leave-one-out cross-validation. The cross-validation root mean squared error was 0.8% kaempferol. In addition to the strong correlation between the percent of kaempferol with pruning weights in this vineyard (FIG. 8D), the percent of kaempferol had a strong correlation with SAGA wetness index (r=−0.48; p<0.001; data not shown) and NDVI (r=−0.60; p<0.001; FIGS. 17B and 17D). In addition, there was a strong correlation between SAGA wetness index and NDVI (r=0.5; p<0.001; data not shown).

After grouping experimental units by their dormant pruning weights into low, medium and high vigor these came as significantly different from each other (FIG. 18). These differences in canopy size did not translate into different total flavonol content. Contrarily, the percent of kaempferol, quercetin, and myricetin flavonols was significantly different in the Low vigor group compared to Medium and High. Furthermore, mean difference in these three parameters was greater from Low to Medium than from Medium to High, suggesting an attenuation in the response to increasing canopy size, similar to the relationship reported in FIG. 8A. This expected response was also observed in Merlot grapevine where leaf or shoot removal was applied (FIG. 18) that had nearly two-fold increase in flavonol content and higher percent of kaempferol and percent of quercetin in detriment of the percent of myricetin, compared to medium vigor untreated controls. Combining shoot thinning and leaf removal affected the percent of kaempferol by increasing its content greater than when leaf removal was applied alone.

### REFERENCES

Acevedo de la Cruz, A., Hilbert, G., Rivière, C., Mengin, V., Ollat, N., Bordenave, L., Decroocq, S., Delaunay, J.-C., Delrot, S., Mérillon, J.-M., Monti, J.-P., Gomès, E. and Richard, T., 2012. Anthocyanin identification and composition of wild Vitis spp. accessions by using LC-MS and LC-NMR. Analytica Chimica Acta, 732(0): 145-152.
Arroyo-Currás, N., Rosas-Garcia, V. M. and Videa, M., 2016. Substituent inductive effects on the electrochemical oxidation of flavonoids studied by square wave voltammetry and Ab initio calculations. Molecules, 21(11): 1422.
Baluja, J., Diago, M. P., Goovaerts, P. and Tardaguila, J., 2012. Assessment of the spatial variability of anthocyanins in grapes using a fluorescence sensor: relationships with vine vigour and yield. Precision Agriculture, 13(4), pp. 457-472.
Bergqvist, J., Dokoozlian, N. and Ebisuda, N., 2001. Sunlight exposure and temperature effects on berry growth and composition of Cabernet Sauvignon and Grenache in the Central San Joaquin Valley of California. American Journal of Enology and Viticulture, 52(1): 1-7.
Bouhsain, Z., Garrigues, S., and de la Guardia, M. (1997). PLS-UV spectrophotometric method for the simultaneous determination of paracetamol, acetylsalicylic acid and caffeine in pharmaceutical formulations. Fresenius. Journal of Analytical Chemistry. 357, 973-976.
Castellarin, S. D. and Di Gaspero, G., 2007. Transcriptional control of anthocyanin biosynthetic genes in extreme phenotypes for berry pigmentation of naturally occurring grapevines. BMC Plant Biology, 7: 46.
Castellarin, S. D., Pfeiffer, A., Sivilotti, P., Degan, M., Peterlunger, E. and Di Gaspero, G., 2007. Transcriptional regulation of anthocyanin biosynthesis in ripening fruits of grapevine under seasonal water deficit. Plant Cell & Environment, 30(11): 1381-1399.
Castillo-Munoz, N., Gómez-Alonso, S., Garcia-Romero, E. and Hermosín-Gutiérrez, I., 2007. Flavonol profiles of Vitis vinifera red grapes and their single-cultivar wines. Journal of Agricultural and Food Chemistry, 55: 11.
Chebil, L., Humeau, C., Falcimaigne, A., Engasser, J.-M. and Ghoul, M., 2006. Enzymatic acylation of flavonoids. Process Biochemistry, 41(11): 2237-2251.
Chellemi, D. O. and Marois, J., 1992. Influence of leaf removal, fungicide applications, and fruit maturity on incidence and severity of grape powdery mildew. American Journal of Enology and Viticulture, 43(1): 53-57.
Chorti, E., Guidoni, S., Ferrandino, A. and Novello, V., 2010. Effect of different cluster sunlight exposure levels on ripening and anthocyanin accumulation in Nebbiolo grapes. American Journal of Enology and Viticulture, 61(1): 23-30.
Cohen, S. D., Tarara, J. M., Gambetta, G. A., Matthews, M. A. and Kennedy, J. A., 2012. Impact of diurnal temperature variation on grape berry development, proanthocyanidin accumulation, and the expression of flavonoid pathway genes. Journal of Experimental Botany, 63(7): 2655-2665.
Cooper, J. E., 2004. Multiple Responses of Rhizobia to Flavonoids During Legume Root Infection, Advances in Botanical Research. Academic Press, pp. 1-62.
Cortell, J. M., Halbleib, M., Gallagher, A. V., Righetti, T. L. and Kennedy, J. A., 2005. Influence of vine vigor on grape (Vitis vinifera L. cv. Pinot noir) and wine proanthocyanidins. Journal of Agricultural and Food Chemistry, 53(14): 5798-5808.
Cortell, J. M. and Kennedy, J. A., 2006. Effect of shading on accumulation of flavonoid compounds in (Vitis vinifera L.) pinot noir fruit and extraction in a model system. Journal of Agricultural and Food Chemistry, 54(22): 8510-20.
de Souza, C. R., Maroco, J. P., dos Santos, T. P., Rodrigues, M. L., Lopes, C. M., Pereira, J. S. and Chaves, M. M., 2003. Partial rootzone drying: regulation of stomatal aperture and carbon assimilation in field-grown grapevines (Vitis vinifera cv. Moscatel). Functional Plant Biology, 30(6): 653-662.
Dixon, R. A., Liu, C. and Jun, J. H., 2013. Metabolic engineering of anthocyanins and condensed tannins in plants. Current Opinion in Biotechnology, 24(2): 329-335.
Downey, M. and Rochfort, S., 2008. Simultaneous separation by reversed-phase high-performance liquid chromatography and mass spectral identification of anthocyanins and flavonols in Shiraz grape skin. Journal of Chromatography A, 1201(1): 43-47.
Downey, M. O., Harvey, J. S. and Robinson, S. P., 2004. The effect of bunch shading on berry development and flavonoid accumulation in Shiraz grapes. Australian Journal of Grape and Wine Research, 10: 19.
Farquhar, G. D., Ehleringer, J. R. and Hubick, K. T., 1989. Carbon Isotope Discrimination and Photosynthesis. Annual Review of Plant Physiology and Plant Molecular Biology, 40: 503-537.
Fournier-Level, A., Hugueney, P., Verries, C., This, P. and Ageorges, A., 2011. Genetic mechanisms underlying the methylation level of anthocyanins in grape (Vitis vinifera L.). BMC Plant Biology, 11: 179.
Gaudillere, J. P., Van Leeuwen, C. and Ollat, N., 2002. Carbon isotope composition of sugars in grapevine, an integrated indicator of vineyard water status. Journal of Experimental Botany, 53(369): 757-63.
Gibberd, M. R., Walker, R. R., Blackmore, D. H. and Condon, A. G., 2001. Transpiration efficiency and carbon—isotope discrimination of grapevines grown under well—watered conditions in either glasshouse or vineyard. Australian Journal of Grape and Wine Research, 7(3): 110-117.
Haselgrove, L., Butting, D., van Heeswijck, R., HØJ, P. B., Dry, P. R., Ford, C. and Land, P. G. I., 2000. Canopy microclimate and berry composition: The effect of bunch exposure on the phenolic composition of Vitis vinifera L cv. Shiraz grape berries. Australian Journal of Grape and Wine Research, 6(2): 141-149.
Hermosín-Gutiérrez, I., Castillo-Muñoz, N., Gómez-Alonso, S. and Garcia-Romero, E., 2011. Flavonol Profiles for Grape and Wine Authentication, Progress in Authentication of Food and Wine. ACS Symposium Series. American Chemical Society, pp. 113-129.
Hilbert, G., Temsamani, H., Bordenave, L., Pedrot, E., Chaher, N., Cluzet, S., Delaunay, J.-C., Ollat, N., Delrot, S., Mérillon, J.-M., Gomès, E. and Richard, T., 2015. Flavonol profiles in berries of wild Vitis accessions using liquid chromatography coupled to mass spectrometry and nuclear magnetic resonance spectrometry. Food Chemistry, 169(Supplement C): 49-58.
Kaltenbach, M., Schröder, G., Schmelzer, E., Lutz, V. and Schröder, J., 1999. Flavonoid hydroxylase from Catharanthus roseus: cDNA, heterologous expression, enzyme properties and cell-type specific expression in plants. Plant Journal, 19(2): 183-193.
Keller, M., 2010. Science of Grapevines: Anatomy and Physiology. Science of Grapevines: Anatomy and Physiology: 1-377.
Kliewer, W. M. and Dokoozlian, N. K., 2005. Leaf area/crop weight ratios of grapevines: Influence on fruit composition and wine quality. American Journal of Enology and Viticulture, 56(2): 170-181.
Koch, A., Ebeler, S. E., Williams, L. E. and Matthews, M. A., 2012. Fruit ripening in Vitis vinifera: light intensity before and not during ripening determines the concentration of 2-methoxy-3-isobutylpyrazine in Cabernet Sauvignon berries. Physiologia Plantarum, 145(2): 275-285.
Koes, R. E., Quattrocchio, F. and Mol, J. N. M., 1994. The flavonoid biosynthetic pathway in plants: Function and evolution. BioEssays, 16(2): 123-132.
Kolb, C. A., Kaser, M. A., Kopecky, J., Zotz, G., Riederer, M. and Pfundel, E. E., 2001. Effects of natural intensities of visible and ultraviolet radiation on epidermal ultraviolet screening and photosynthesis in grape leaves. Plant Physiology, 127(3): 863-75.
Kong, C., Xu, X., Zhou, B., Hu, F., Zhang, C. and Zhang, M., 2004. Two compounds from allelopathic rice accession and their inhibitory activity on weeds and fungal pathogens. Phytochemistry, 65(8): 1123-1128.
Koundouras, S., Tsialtas, I. T., Zioziou, E. and Nikolaou, N., 2008. Rootstock effects on the adaptive strategies of grapevine (Vitis vinifera L. cv. Cabernet-Sauvignon) under contrasting water status: leaf physiological and structural responses. Agriculture, Ecosystems & Environment, 128(1-2): 86-96.
Lagrange, H., Jay-Allgmand, C. and Lapeyrie, F., 2001. Rutin, the phenolglycoside from eucalyptus root exudates, stimulates Pisolithus hyphal growth at picomolar concentrations. New Phytologist, 149(2): 349-355.
Malacarne, G., Costantini, L., Coller, E., Battilana, J., Velasco, R., Vrhovsek, U., Grando, M. S. and Moser, C., 2015. Regulation of flavonol content and composition in (Syrah×Pinot Noir) mature grapes: integration of transcriptional profiling and metabolic quantitative trait locus analyses. Journal of Experimental Botany, 66(15): 4441-4453.
Martínez-Lüscher, J., Chen, C. C. L., Brillante, L. and Kurtural, S. K., 2017. Partial solar radiation exclusion with color shade nets reduce the degradation of organic acids and flavonoids of grape berry (Vitis vinifera L.). Journal of Agricultural and Food Chemistry.
Martínez-Lüscher, J., Hilbert, G., Sánche-Díaz, M., Delrot, S., Aguirreolea, J., Pascual, I. and Gomès, E., 2013a. UV-B radiation and water deficit interaction modulates the synthesis of grape berry skin flavonols through ripening. In: Abstracts of The 2nd Network meeting of COST Action FA0906 (UV4growth). pp. 41 ISBN 978-80-904351-7-9.
Martínez-Lüscher, J., Morales, F., Delrot, S., Sánchez-Díaz, M., Gomès, E., Aguirreolea, J. and Pascual, I., 2013b. Short- and long-term physiological responses of grapevine leaves to UV-B radiation. Plant Science, 213(0): 114-122.
Martínez-Lüscher, J., Sánchez-Díaz, M., Delrot, S., Aguirreolea, J., Pascual, I. and Gomès, E., 2014a. Ultraviolet-B radiation and water deficit interact to alter flavonol and anthocyanin profile in grapevine berries through transcriptomic regulation. Plant and Cell Physiology, 55(11): 1925-1936.
Martínez-Lüscher, J., Torres, N., Hilbert, G., Richard, T., Sánchez-Díaz, M., Delrot, S., Aguirreolea, J., Pascual, I. and Gomès, E., 2014b. Ultraviolet-B radiation modifies the quantitative and qualitative profile of flavonoids and amino acids in grape berries. Phytochemistry, 102(0): 106-114.
Mattivi, F., Guzzon, R., Vrhovsek, U., Stefanini, M. and Velasco, R., 2006. Metabolite profiling of grape: Flavonols and anthocyanins. Journal of Agricultural and Food Chemistry, 54(20): 7692-702.
Matus, J. T., Loyola, R., Vega, A., Pena-Neira, A., Bordeu, E., Arce-Johnson, P. and Alcalde, J. A., 2009. Post-veraison sunlight exposure induces MYB-mediated transcriptional regulation of anthocyanin and flavonol synthesis in berry skins of Vitis vinifera. Journal of Experimental Botany, 60(3): 853-67.
Nakabayashi, R., Yonekura-Sakakibara, K., Urano, K., Suzuki, M., Yamada, Y., Nishizawa, T., Matsuda, F., Kojima, M., Sakakibara, H., Shinozaki, K., Michael, A. J., Tohge, T., Yamazaki, M. and Saito, K., 2014. Enhancement of oxidative and drought tolerance in Arabidopsis by overaccumulation of antioxidant flavonoids. The Plant Journal, 77(3): 367-379.
Pastore, C., Zenoni, S., Fasoli, M., Pezzotti, M., Tornielli, G. B. and Filippetti, I., 2013. Selective defoliation affects plant growth, fruit transcriptional ripening program and flavonoid metabolism in grapevine. BMC Plant Biology, 13: 30.
Reshef, N., Walbaum, N., Agam, N. and Fait, A., 2017. Sunlight Modulates Fruit Metabolic Profile and Shapes the Spatial Pattern of Compound Accumulation within the Grape Cluster. Frontiers in Plant Science, 8: 70.
Richardson, A. D., Reeves III, J. B., and Gregoire, T. G. (2004). Multivariate analyses of visible/near infrared (VIS/NIR) absorbance spectra reveal underlying spectral differences among dried, ground conifer needle samples from different growth environments. New Phytologist. 161, 291-301.
Ryona, I., Pan, B. S., Intrigliolo, D. S., Lakso, A. N. and Sacks, G. L., 2008. Effects of cluster light exposure on 3-Isobutyl-2-methoxypyrazine accumulation and degradation patterns in red wine grapes (Vitis vinifera L. cv. Cabernet Franc). Journal of Agricultural and Food Chemistry, 56(22): 10838-10846.
Sadilova, E., Carle, R. and Stintzing, F. C., 2007. Thermal degradation of anthocyanins and its impact on color and in vitro antioxidant capacity. Molecular Nutrition & Food Research, 51(12): 1461-1471.
Steyn, J., Aleixandre-Tudó, J. L. and Aleixandre, J. L., 2016. Grapevine Vigour and Within-Vineyard Variability: a Review. growth, 18(23): 24.
Sweetman, C., Sadras, V. O., Hancock, R. D., Soole, K. L. and Ford, C. M., 2014. Metabolic effects of elevated temperature on organic acid degradation in ripening Vitis vinifera fruit. Journal of Experimental Botany, 65(20): 5975-5988.
Tabart, J., Kevers, C., Pincemail, J., Defraigne, J.-O. and Dommes, J., 2009. Comparative antioxidant capacities of phenolic compounds measured by various tests. Food Chemistry, 113(4): 1226-1233.
Tarara, J. M., Lee, J., Spayd, S. E. and Scagel, C. F., 2008. Berry temperature and solar radiation alter acylation, proportion, and concentration of anthocyanin in Merlot grapes. American Journal of Enology and Viticulture, 59(3): 235-247.
Vogt, T. and Jones, P., 2000. Glycosyltransferases in plant natural product synthesis: characterization of a supergene family. Trends in Plant Science, 5(9): 380-386.
Wickman, F. E., 1952. Variations in the Relative Abundance of the Carbon Isotopes in Plants. Nature, 169: 1051.
Winkel, B. S., 2004. Metabolic channeling in plants. Annual Review of Plant Biology, 55: 85-107.
Wold, S., Sjöström, M. and Eriksson, L., 2001. PLS-regression: a basic tool of chemometrics. Chemometrics and Intelligent Laboratory Systems, 58(2): 109-130.
Zhao, C.-L., Yu, Y.-Q., Chen, Z.-J., Wen, G.-S., Wei, F.-G., Zheng, Q., Wang, C.-D. and Xiao, X.-L., 2017. Stability-increasing effects of anthocyanin glycosyl acylation. Food Chemistry, 214(Supplement C): 119-128.

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A method for determining whether a grape is under exposed or overexposed to solar radiation, comprising:
(a) extracting and purifying flavonols comprising 4′-OH flavonol, 3′4′-OH flavonol, and 3′4′5′-OH flavonol from grape skins;
(b) determining percentages of 4′-OH flavonol, 3′4′-OH flavonol, and 3′4′5′-OH in the extracted flavonols using a spectrophotometer and a Partial Least Squares (PLS) regression model, wherein:
if the percentage of 4′-OH flavonol content is below 4%, then the grapes are underexposed and canopy will need pruning; or
if the percentage of 4′-OH flavonol is above 10.5%, then the grapes are overexposed and need cover; or
if 3′4′5′-OH flavonol content is above 45%, then the grapes are underexposed and canopy will need pruning; or
if the percentage of 3′4′5′-OH flavonol is below 30%, then the grapes are overexposed and need cover.

2. The method of claim 1, wherein the PLS regression model comprises an equation using the absorbance of a purified flavonol mixture at multiple wavelengths between 220 and 800 nm.

3. The method of claim 1, wherein the PLS model calculates the concentration of each of 4′-OH flavonol, 3′4′-OH flavonol, and 3′4′5′-OH flavonol, calculates their percentage of total flavonol and plots the percentages in a ternary plot for the comparison to grapes with different exposures.

4. The method of claim 1, wherein pruning the grape canopy comprises shoot thinning and/or leaf removal to increase exposure.

5. A computer program product comprising a computer-readable storage medium containing computer program code for:
receiving a data set representing the absorbance of one or more flavonols measured at one or more wavelengths between 220 and 800 nm,
applying a Partial Least Squares (PLS) regression to generate percentage of the one or more flavonols selected from the group consisting of 4′-OH flavonol, 3′4′-OH flavonol, and 3′4′5′-OH, and
comparing the percentage of the flavonol to a cut-off percentage.

6. The computer program product of claim 5, wherein the flavonol is 4′-OH flavonol and the cut-off percentage is 4% out of total flavonol content.

7. The computer program product of claim 5, wherein the flavonol is 4′-OH flavonol and the cut-off percentage is 10.5% out of total flavonol content.

8. The computer program product of claim 5, wherein the flavonol is 3′4′5′-OH flavonol and the cut-off percentage is 30% out of total flavonol content.

9. The computer program product of claim 5, wherein the flavonol is 3′4′5′-OH flavonol and the cut-off percentage is 45% out of total flavonol content.

10. The computer program product of claim 5, further comprising code for indicating whether pruning is suggested or not based on the comparing.
